(19) United States
(12) Patent Application Publication
(10) Pub. No.: US 2024/0295545 A1
(43) Pub. Date: Sep. 5, 2024

(54) GLIOMA THERAPY

(71) Applicant: Imperial College Innovations Limited, London (GB)

(72) Inventors: Valentin K. Gribkoff, South Kensington, London (GB); Kambiz N. Alavian, South Kensington, London (GB); Travis S. Tierney, South Kensington, London (GB)

(21) Appl. No.: 18/276,956

(22) PCT Filed: Feb. 11, 2022
(86) PCT No.: PCT/GB2022/050376

§ 371 Date: Aug. 11, 2023

(30) Foreign Application Priority Data
Feb. 11, 2021 (GB) 2101933.6

(51) Int. Cl. G01N 33/50; A61K 31/075; A61K 31/381; A61K 31/423; A61K 31/426; A61K 31/44; A61K 31/495; A61P 35/00; G01N 21/64

(52) U.S. Cl. CPC G01N 33/5079; A61K 31/075; A61K 31/381; A61K 31/423; A61K 31/426; A61K 31/44; A61K 31/495; A61P 35/00; G01N 21/6428; G01N 33/5058; G01N 2500/10

(57) ABSTRACT

The invention is directed to a method for identifying a candidate anti-glioma drug that is a mitochondrial F₁Fₒ ATP Synthase c-subunit leak channel opener, the method comprising: a. providing an admixture comprising: i. a candidate drug; ii. a submitochondrial vesicle (SMV) preparation; and iii. an H⁺ probe, wherein the H⁺ probe remains outside of the SMV(s); b. contacting the admixture with ATP; c. measuring a level of H⁺ outside of the SMV(s) via the H⁺ probe; d. comparing the level of H⁺ at step c) with a level of H⁺ in a control admixture lacking the candidate drug; and identifying the drug as a candidate anti-glioma drug that is a mitochondrial F₁Fₒ ATP Synthase c-subunit leak channel opener, when the level of H⁺ is higher compared to the level of H⁺ in the control admixture; or identifying that the drug is not a candidate anti-glioma drug that is a mitochondrial F₁Fₒ ATP Synthase c-subunit leak channel opener, when the level of H⁺ is the same or lower compared to the level of H⁺ in the control admixture.

The present invention relates to therapy for glioma, as well as methods for screening for anti-glioma drugs and identifying responder patients.

### BACKGROUND

Glioma is a type of tumor that occurs in the brain and spinal cord. Gliomas comprise about 30% of all primary brain tumours and 80% of all malignant brain tumours. The median survival time after diagnosis for glioblastoma multiforme (GBM), the most aggressive form of the disease, is about 12 months and GBM still has one of the worst 5-year survival rates among all cancers.

The current standard of care, in addition to radiation, is temozolomide (TMZ), an agent which reduces proliferation in some patients. TMZ is a prodrug which exerts its effects by methylating DNA at a particular guanine residue, leading to genomic instability due to mismatched pairing of guanine with thymine, and cell death. TMZ is effective in about 50% of glioma patients. However, the mismatch is repaired in some patients e.g. via the sequestering of methylated nucleotide by the enzyme O⁶ methylguanine-DNA methyltransferase (MGMT), and methylation of the enzyme promoter MGMT is one of the strongest predictors of response to TMZ. This results in a proportion of patients who are effectively refractory to TMZ treatment, and there is therefore a particular need for new therapies for a glioma patient cohort suffering TMZ resistance. These issues are compounded by the relatively few alternative therapies, due at least in part to a lack of understanding of molecular targets against which candidate therapies may be screened. As such, there exists a need in the art for anti-glioma therapies (ideally effective in even those patients that are TMZ-refractory), as well as methods to identify such therapies and (subsequently) the most effective therapy for any given patient e.g. in a personalised manner.

The present invention overcomes one or more of the above-mentioned problems.

### SUMMARY OF THE INVENTION

The present invention is predicated on the surprising demonstration that there exists glioma populations having particularly efficient mitochondria which fuels high levels of proliferation and malignancy. Building on this demonstration, the inventors provide a unique therapeutic strategy (outlined in more detail below) for treating glioma through perturbing mitochondrial coupling by molecular targeting of the “F₁Fₒ ATP Synthase c-subunit leak channel”, inducing proton leakage (H+) and thus suppressing mitochondrial efficiency that fuels growth in these particularly proliferative gliomas. Having demonstrated this unique clinical situation, assays for measuring proton leakage have been adapted to provide rapid, sensitive methods for screening drug libraries for candidate anti-glioma drugs for input to clinical trial assessment. These findings are elaborated on below.

In more detail, the proliferation of cancer cells (e.g. glioma) depends on mitochondria for the de novo synthesis of ATP and production of macromolecules such as lipids, proteins and nucleotides. The production of ATP through mitochondrial OXPHOS and synthesis of the intermediates of the tricarboxylic acid (TCA), required for anabolic growth, are both reliant upon an electrochemical gradient across the mitochondrial inner membrane. The mitochondrial membrane potential is mainly a function of the activity of the electron transport chain and OXPHOS enzymes as well as the mitochondrial inner membrane ion leak currents. It has previously been demonstrated that enhanced mitochondrial coupling and efficiency of ATP synthesis involves closure of a specific inner membrane “leak channel” within the F₁Fₒ ATP synthase of the mitochondria (Alavian, K. N. et al.; Proc Natl Acad Sci USA. 2014 Jul. 22; 111(29): 10580-10585; Alavian, K. N. et al.; Nat Cell Biol 13, 1224-1233, doi:10.1038/ncb2330 (2011)).

To investigate whether there exist glioma populations that could be suppressed by targeting mitochondrial coupling, the inventors compared the metabolic and mitochondrial energetic profile of cells from a slow-growing glioma/astrocytoma to that of highly proliferative glioma primary tumour cells by performing ‘hypoxia’ experiments with primary tumour cells, in which primary tumour cells were deprived of adequate oxygen supply and compared with control cells not so deprived (‘normoxia’). Interestingly, it was found that the rate of proliferation in the highly proliferative glioma population was significantly reduced under hypoxia, suggesting that they rely more on their mitochondria than less proliferative gliomas (see Example 1 and FIG. 1A). Furthermore, it was demonstrated that toxins which act by inhibiting mitochondrial respiratory complexes and oxidative phosphorylation, impeded the proliferation of cells from a highly proliferative glioma population, without a significant effect on the less proliferative glioma population (FIG. 1B). A direct correlation was observed between mitochondrial membrane potential (measured by TMRE) and the rate of cellular proliferation, demonstrating that highly proliferative glioma cells employ efficient mitochondrial metabolism to achieve their higher proliferation rates (FIG. 2).

These results are supportive of a therapeutic approach for treatment of highly proliferative glioma tumours via modulation of mitochondrial leak channels (to induce proton leakage) to perturb mitochondrial coupling, a candidate channel being the F₁Fₒ ATP synthase c-subunit leak channel. The inventors thus investigated whether modulation of this leak channel (inducing leakage) causes glioma suppression. Having identified candidate anti-glioma drugs that induce H+ leakage through the mitochondrial F₁Fₒ ATP Synthase c-subunit leak channel (via a fluorescence-based screen outlined in detail in Example 2), the inventors proceeded to validate the suppressive effect of these drugs on isolated cells obtained from patient glioma biopsies. In more detail, the inventors examined the effect of the positive hits (from said screen), as well as temozolomide, TMZ (the standard chemotherapeutic agent for treatment of adult and paediatric gliomas), on proliferation of GBM cells in vitro. In this hit confirmation assay, 35 of the 40 drugs reduced the proliferation of “GBM1” cells by 12-96% (n=4, p<0.01) (FIG. 4B). These results demonstrate that the drug hits (which induce H+ leakage through the mitochondrial F₁Fₒ ATP Synthase c-subunit leak channel) reduced the proliferation of highly proliferative glioma populations (FIG. 4C). Interestingly, TMZ was effective in slowing down proliferation of a subset of GBM cell lines and had no effect on the cell line (GBM1) with the highest rate of proliferation, while selected candidate drugs significantly reduced the rate of proliferation in this specific cell line (FIG. 4D,E). These results demonstrate that metabolic reprogramming through modulation of mitochondrial ion leak currents (as a standalone therapy and/or as part of a combination therapy) to be an effective method for treatment of malignant glioma. Furthermore, this two-step method (e.g. fluorescence-based screen plus glioma suppression assays) allows identification of the most effective pharmacological agents and establishes a pathway for optimal treatment of highly proliferative gliomas.

In summary, the above described (surprising) technical effects support the advantageous ability of a leak channel opener (e.g. medicament that targets the mitochondrial F₁Fₒ ATP Synthase c-subunit leak channel, inducing H⁺ leakage) to treat highly proliferative glioma populations, which represent a patient subgroup uniquely suited to receiving the therapy described herein. In other words, the previously unrecognised technical effect (glioma suppression via inducing proton leakage through the F₁Fₒ ATP Synthase c-subunit leak channel) identifies a new clinical situation.

### DETAILED DESCRIPTION

Broad aspects of the invention are directed to any of:
a medicament that targets the mitochondrial F₁Fₒ ATP Synthase c-subunit leak channel for use in a method of suppressing a glioma in a patient;
a method of suppressing a glioma in a patient, the method comprising administering a medicament that targets the mitochondrial F₁Fₒ ATP Synthase c-subunit leak channel to the patient.

Broad aspects of the invention are directed to any of:
a medicament comprising a mitochondrial F₁Fₒ ATP Synthase c-subunit leak channel opener for use in a method of suppressing a glioma in a patient;
a method of suppressing a glioma in a patient, the method comprising administering a medicament comprising a mitochondrial F₁Fₒ ATP Synthase c-subunit leak channel opener to the patient.

The term a “medicament that targets the F₁Fₒ ATP synthase c-subunit” as referred to herein may be used interchangeably throughout this specification with the term “F₁Fₒ ATP synthase c-subunit leak channel opener”, the term “modulator of the F₁Fₒ ATP synthase c-subunit leak channel” and/or the term “medicament comprising a mitochondrial F₁Fₒ ATP Synthase c-subunit leak channel opener”. These four terms may be used interchangeably throughout, and refer to a medicament (or leak channel opener or modulator) that promotes proton (H+) leakage through the mitochondrial F₁Fₒ ATP Synthase c-subunit leak channel (e.g. to cause suppression of glioma proliferation); or in other words, that induces proton (H+) leakage through the mitochondrial F₁Fₒ ATP Synthase c-subunit leak channel.

The medicament (or leak channel opener or modulator) is a composition or substance and, as will be discussed below, may be referred to as a “drug”.

A channel modulator (e.g. ion channel modulator) is a type of medicament (e.g. drug) which modulates ion channels. Such channel modulators include channel blockers and channel openers. The present invention is directed to the latter, for example channel openers that induce proton (H+) leakage through the mitochondrial F₁Fₒ ATP Synthase c-subunit leak channel. For example, a medicament/channel opener/modulator described herein may positively modulate e.g. activate the F₁Fₒ ATP Synthase c-subunit leak channel.

Broad aspects of the invention may be described as:
an F₁Fₒ ATP synthase c-subunit leak channel opener for use in a method of suppressing a glioma in a patient;
a method of suppressing a glioma in a patient, the method comprising administering an F₁Fₒ ATP synthase c-subunit leak channel opener to the patient.

Broad aspects of the invention may be described as:
a modulator (e.g. positive modulator) of the F₁Fₒ ATP synthase c-subunit leak channel for use in a method of suppressing a glioma in a patient;
a method of suppressing a glioma in a patient, the method comprising administering a modulator (e.g. positive modulator) of the F₁Fₒ ATP synthase c-subunit leak channel to the patient.

Preferably, any one of the following embodiments may apply to said broad aspect(s):
following contact with a glioma cell in the patient the medicament induces: proton (H+) leakage through the mitochondrial F₁Fₒ ATP Synthase c-subunit leak channel (e.g. to cause suppression of glioma proliferation);
following contact with a glioma cell in the patient the medicament promotes: proton (H+) leakage through the mitochondrial F₁Fₒ ATP Synthase c-subunit leak channel (e.g. to cause suppression of glioma proliferation);
the medicament suppresses the glioma by inducing proton (H+) leakage through the mitochondrial F₁Fₒ ATP Synthase c-subunit leak channel (e.g. to cause suppression of glioma proliferation);
the medicament suppresses the glioma by promoting proton (H+) leakage through the mitochondrial F₁Fₒ ATP Synthase c-subunit leak channel (e.g. to cause suppression of glioma proliferation).

Preferably, any one of the following embodiments may apply to said broad aspect(s):
following contact with a glioma cell in the patient the leak channel opener induces: proton (H+) leakage through the mitochondrial F₁Fₒ ATP Synthase c-subunit leak channel (e.g. to cause suppression of glioma proliferation);
following contact with a glioma cell in the patient the leak channel opener promotes: proton (H+) leakage through the mitochondrial F₁Fₒ ATP Synthase c-subunit leak channel (e.g. to cause suppression of glioma proliferation);
the leak channel opener suppresses the glioma by inducing proton (H+) leakage through the mitochondrial F₁Fₒ ATP Synthase c-subunit leak channel (e.g. to cause suppression of glioma proliferation);
the leak channel opener suppresses the glioma by promoting proton (H+) leakage through the mitochondrial F₁Fₒ ATP Synthase c-subunit leak channel (e.g. to cause suppression of glioma proliferation).

In one aspect, the present invention provides a mitochondrial F₁Fₒ ATP Synthase c-subunit leak channel opener for use in a method of suppressing a glioma in a patient, wherein following contact with a glioma cell in the patient the leak channel opener induces:
proton (H+) leakage through the mitochondrial F₁Fₒ ATP Synthase c-subunit leak channel.

One aspect of the invention provides a mitochondrial F₁Fₒ ATP Synthase c-subunit leak channel opener for use in a method of suppressing a glioma in a patient, by inducing proton (H+) leakage through the mitochondrial F₁Fₒ ATP Synthase c-subunit leak channel.

One aspect of the invention provides a mitochondrial F₁Fₒ ATP Synthase c-subunit leak channel opener for use in inducing proton (H+) leakage through the mitochondrial F₁Fₒ ATP Synthase c-subunit leak channel to suppress a glioma in a patient.

One aspect of the invention provides a mitochondrial F₁Fₒ ATP Synthase c-subunit leak channel opener for use in a method of suppressing a glioma in a patient, wherein following contact with a glioma cell in the patient the leak channel opener induces:
proton (H+) leakage through the mitochondrial F₁Fₒ ATP Synthase c-subunit leak channel, causing suppression of glioma proliferation.

In one aspect of the invention, there is provided a method of suppressing a glioma in a patient, the method comprising administering to the patient a mitochondrial F₁Fₒ ATP Synthase c-subunit leak channel opener, wherein following administration the leak channel opener induces, in the glioma:
proton (H+) leakage through the mitochondrial F₁Fₒ ATP Synthase c-subunit leak channel.

One aspect of the invention provides a method of suppressing a glioma in a patient, the method comprising administering to the patient a mitochondrial F₁Fₒ ATP Synthase c-subunit leak channel opener, wherein following administration the leak channel opener induces, in the glioma:
proton (H+) leakage through the mitochondrial F₁Fₒ ATP Synthase c-subunit leak channel causing suppression of glioma proliferation.

Preferably, the leak channel opener (e.g. medicament) may induce a reduction in mitochondrial membrane potential in cells of the glioma (e.g. when compared to mitochondrial membrane potential in cells of the glioma pre-administration of the leak channel opener; or when compared to mitochondrial membrane potential in cells of the glioma in the absence of the leak channel opener).

One aspect of the invention provides a mitochondrial F₁Fₒ ATP synthase c-subunit leak channel opener, for use in a method of suppressing a glioma;
wherein cells of the glioma have a mitochondrial transmembrane potential (ΔΨm) that is higher than a mitochondrial transmembrane potential in non-cancerous glial cells, and
wherein the leak channel opener decreases the mitochondrial transmembrane potential in said cells of the glioma (e.g. by inducing H⁺ leakage through the mitochondrial F₁Fₒ ATP Synthase c-subunit leak channel).

One aspect of the invention provides a mitochondrial F₁Fₒ ATP synthase c-subunit leak channel opener, for use in a method of suppressing a glioma associated with aberrant mitochondrial activity;
wherein cells of the glioma have a mitochondrial transmembrane potential (ΔΨm) that is higher than a mitochondrial transmembrane potential in non-cancerous glial cells, and
wherein the leak channel opener decreases the mitochondrial transmembrane potential in said cells of the glioma (e.g. by inducing H⁺ leakage through the mitochondrial F₁Fₒ ATP Synthase c-subunit leak channel).

One aspect of the invention provides a mitochondrial F₁Fₒ ATP synthase c-subunit leak channel opener, for use in a method of suppressing a glioma;
wherein cells of the glioma have a mitochondrial transmembrane potential (ΔΨm) that is higher than a mitochondrial transmembrane potential in cells of a glioma that is not suppressed by the leak channel opener, and
wherein the leak channel opener decreases the mitochondrial transmembrane potential in said cells of the glioma (e.g. by inducing H⁺ leakage through the mitochondrial F₁Fₒ ATP Synthase c-subunit leak channel).

One aspect of the invention provides a mitochondrial F₁Fₒ ATP synthase c-subunit leak channel opener, for use in a method of suppressing a glioma associated with aberrant mitochondrial activity;
wherein cells of the glioma have a mitochondrial transmembrane potential (ΔΨm) that is higher than a mitochondrial transmembrane potential in cells of a glioma that is not suppressed by the leak channel opener, and
wherein the leak channel opener decreases the mitochondrial transmembrane potential in said cells of the glioma in the patient (e.g. by inducing (aka promoting) H⁺ leakage through the mitochondrial F₁Fₒ ATP Synthase c-subunit leak channel).

In a related aspect, there is provided a method of suppressing a glioma (e.g. associated with aberrant mitochondrial activity) in a patient, the method comprising administering a mitochondrial F₁Fₒ ATP synthase c-subunit leak channel opener to the patient;
wherein cells of the glioma have a mitochondrial transmembrane potential (ΔΨm) that is higher than a mitochondrial transmembrane potential in non-cancerous glial cells, and
wherein the leak channel opener decreases the mitochondrial transmembrane potential in said cells of the glioma (e.g. by inducing H⁺ leakage through the mitochondrial F₁Fₒ ATP Synthase c-subunit leak channel).

As outlined above, the inventors have demonstrated that a leak channel opener (e.g. medicament) of the invention may provide for suppression of such glioma by inducing H⁺ leakage through the mitochondrial F₁Fₒ ATP Synthase c-subunit leak channel.

An “F₁Fₒ ATP synthase” is a multimeric protein complex (of the mitochondrial inner membrane in eukaryotes). A principle function of this complex in the cell is to convert energy of an H+ electrochemical gradient across the membrane into energy of chemical bonds in ATP molecules by coupling of H+ transfer and ATP synthesis. ATP synthases are present in inner mitochondria membranes in eukaryotes (the plasma membrane in archaea or bacteria). The Fₒ subdomain includes subunits a, b, c, d, F6, e, f, g, A6L, 6.8PL, DAPIT (diabetes-associated protein in insulin sensitive tissues) and OSCP (oligomycin sensitivity-conferring protein); and the F₁ subdomain includes subunits α, β, γ, δ, ε and the regulatory protein IF_{1,} which are located in the mitochondrial matrix. Said c-subunits (of Fₒ) form a “c-subunit ring” that is completely embedded in the membrane (e.g. inner membrane), and provides a leak channel through which protons can be expelled from the mitochondria. Opening of the leak channels in the inner membrane generally cause metabolic inefficiency, and it has been postulated that leak channel activity (e.g. channel closure) is modulated to adjust metabolism and ensure survival even in healthy cells. The work of the present inventors has demonstrated that the proliferation of highly malignant glioma cells (having particularly efficient mitochondria) may be perturbed by opening the leak channels, reducing the metabolic efficiency in these cells. Without wishing to be bound by theory, it is believed that a leak channel opener (e.g. medicament) of the invention causes the c-subunit ring to undergo a measurable conformational change by enlarging its size upon activation of the channel, with this conformational change contributing to proton leakage. As such, the invention involves increasing the “leakiness” of the c-subunit leak channel to quench/suppress glioma proliferation by inhibiting mitochondrial efficiency in highly proliferative populations.

The term “targets the F₁Fₒ ATP synthase c-subunit” embraces direct targeting and indirect targeting of a c-subunit polypeptide. For example, the former (direct targeting) may include the situation in which the leak channel opener (e.g. medicament) interacts with a c-subunit polypeptide directly, inducing leakage. Similarly, the latter (indirect targeting) may include the situation where the leak channel opener (e.g. medicament) targets a non-c-subunit polypeptide (e.g. b-subunit polypeptide) yet leads to induction of proton leakage through the c-subunit leak channel.

As mentioned above, term a “medicament that targets the F₁Fₒ ATP synthase c-subunit” as referred to herein may be used interchangeably throughout this specification with the term “F₁Fₒ ATP synthase c-subunit leak channel opener”, the term “modulator of the F₁Fₒ ATP synthase c-subunit leak channel” and/or the term “medicament comprising a mitochondrial F₁Fₒ ATP Synthase c-subunit leak channel opener”. These four terms may be used interchangeably throughout, and refer to a medicament (or leak channel opener or modulator) that promotes proton (H+) leakage through the mitochondrial F₁Fₒ ATP Synthase c-subunit leak channel (e.g. to cause suppression of glioma proliferation); or in other words, that induces proton (H+) leakage through the mitochondrial F₁Fₒ ATP Synthase c-subunit leak channel.

The person skilled understands that the term “leakage” refers to expulsion of protons from the mitochondria (for example, even where a channel is a two-way channel, leakage refers to the route exiting the mitochondria e.g. across the inner membrane). Thus, for the avoidance of doubt, the term “leakage” in the context of “proton leakage through the mitochondrial F₁Fₒ ATP Synthase c-subunit leak channel” is intended to mean leakage of protons out of the mitochondria.

By inducing such proton leakage (via the F₁Fₒ ATP synthase c-subunit leak channel), the mitochondrial transmembrane potential in cells of the glioma may be reduced. For example, the inventors have demonstrated that highly proliferative glioma populations have a mitochondrial transmembrane potential that is higher than a mitochondrial transmembrane potential in non-cancerous glial cells; or higher than a mitochondrial transmembrane in glioma cells that are not suppressed by the leak channel opener.

Thus, a glioma (to be suppressed) as referred to herein is a glioma that can be suppressed by inducing H⁺ leakage through the mitochondrial F₁Fₒ ATP Synthase c-subunit leak channel.

Glial cells (also known as neuroglia), are non-neuronal cells in the central nervous system (brain and spinal cord) and the peripheral nervous system. It is known that certain types of glial cells can become cancerous, giving rise to glioma. Glial cells (e.g. healthy glial cells) which have not become cancerous are thus “non-cancerous glial cells”. At least three types of glial cells are known to give rise to glioma:
ependymal cells (e.g. ependymocytes);
astrocytes;
oligodendrocytes.

When comparing a glioma to a non-cancerous glial cell herein, the non-cancerous glial cell is preferably of the type from which the glioma is derived. For example:
when the glioma is an ependymoma, the non-cancerous glial cell is preferably an ependymal cell;
when the glioma is an astrocytoma (e.g. glioblastoma multiforme), the non-cancerous glial cell is preferably an astrocyte;
when the glioma is an oligodendroglioma, the non-cancerous glial cell is preferably an oligodendrocyte.

The mitochondrial membrane potential (ΔΨm) refers to a gradient of the electric potential on the inner mitochondrial membrane. Δ m is generated by proton pumps (Complexes I, III and IV) and is a component of the process for energy storage during oxidative phosphorylation. ΔΨm (together with the proton gradient (ΔpH)) forms the transmembrane potential of hydrogen ions which is harnessed to make adenosine triphosphate (ATP). ΔΨm reflects the process of electron transport and oxidative phosphorylation, the driving force behind ATP production, and is thus an indicator of mitochondrial activity.

In more detail, mitochondria provide the majority of a cell's ATP (the main source of energy for metabolism) by a process known as oxidative phosphorylation. This process involves active transfer of positively charged protons across the mitochondrial inner membrane resulting in a net internal negative charge, which is thus known as the mitochondrial transmembrane potential (ΔΨm). The proton gradient is then used by ATP synthase to produce ATP by fusing adenosine diphosphate and free phosphate.

In a first cell that has a ΔΨm that is higher than the Am in a (different) second cell, the net charge across the inner membrane in mitochondria of the first cell is ‘more negative’ (e.g. more polarised) than the corresponding charge across said second cell. The level of the net negative charge across the inner membrane can be detected/quantified by staining cells with positively charged dyes, such as tetramethylrhodamine ethyl ester (TMRE). TMRE emits a red fluorescence e.g. detectable by fluorescence microscopy and the level of TMRE fluorescence in stained cells correlates with the level of polarisation across the inner membrane, and can be used to determine whether mitochondria in a cell have high or low ΔΨm (e.g. due to higher or lower levels of fluorescence in stained cells, respectively).

In one embodiment, cells of the glioma have a mitochondrial transmembrane potential (ΔΨm) that is at least 10% higher than a mitochondrial transmembrane potential in non-cancerous glial cells. For example, the ΔΨm in cells of the glioma may be at least 15%, 20%, 25%, 30%, 35%, 40% or 45% (preferably at least 30%) higher, than ΔΨm in non-cancerous glial cells. For example, the ΔΨm in cells of the glioma may be at least 50%, 100%, 150%, 200%, 250%, 300%, 350% or 400% (preferably at least 300%) higher, than ΔΨm in non-cancerous glial cells. In some embodiments, said higher ΔΨm may be identified by a fold change in ΔΨm. In one embodiment, the ΔΨm in cells of the glioma may be at least about 1.5-fold, 2-fold, 2.5-fold, 3-fold, 3.5-fold, 4-fold, 4.5-fold or 5-fold (preferably at least 4-fold) higher, than ΔΨm in non-cancerous glial cells.

In one embodiment, cells of the glioma have a mitochondrial transmembrane potential (ΔΨm) that is at least 10% higher than a mitochondrial transmembrane potential in cells of a glioma that is not suppressed by the leak channel opener (e.g. medicament). For example, the ΔΨm in cells of the glioma may be at least 15%, 20%, 25%, 30%, 35%, 40% or 45% (preferably at least 30%) higher, than ΔΨm in cells of a glioma that is not suppressed by the leak channel opener. For example, the ΔΨm in cells of the glioma may be at least 50%, 100%, 150%, 200%, 250%, 300%, 350% or 400% (preferably at least 300%) higher, than ΔΨm in cells of a glioma that is not suppressed by the leak channel opener (e.g. medicament). In some embodiments, said higher ΔΨm may be identified by a fold change in ΔΨm. In one embodiment, the ΔΨm in cells of the glioma may be at least about 1.5-fold, 2-fold, 2.5-fold, 3-fold, 3.5-fold, 4-fold, 4.5-fold or 5-fold (preferably at least 4-fold) higher, than ΔΨm in cells of a glioma that is not suppressed by the leak channel opener (e.g. medicament).

Reference to the ΔΨm in cells of the glioma may suitably mean an average ΔΨm in glioma cells of the patient. Similarly, reference to the ΔΨm in non-cancerous glial cells may suitably refer to an average ΔΨm of a population of non-cancerous glial cells. For example, the ΔΨm (of glioma cells and/or non-cancerous glial cells) may be derived by pooling a level of ΔΨm obtained from multiple cells, and calculating an average (for example, mean or median) level of ΔΨm. Thus, the ΔΨm may reflect the average level of ΔΨm in a tumor (e.g. having multiple glioma cells).

Mitochondrial transmembrane potential (ΔΨm) may suitably be measured (e.g. and compared) by TMRE, e.g. a TMRE assay.

Cells of a glioma that have a ΔΨm that is higher than a ΔΨm in non-cancerous glial cells may be identifiable by an assay comprising:
incubating a test sample comprising cells (e.g. isolated cells) of the glioma (e.g. glioma to be treated) in the presence of TMRE, wherein the TMRE is provided at a concentration of 10 nM in the test sample, wherein the test sample is incubated at 37° C.;
comparing a level of TMRE fluorescence (using excitation and emission wavelengths of 550 nm and 575 nm, respectively) in the test sample with a level of TMRE fluorescence in a control sample comprising non-cancerous glial cells; and
confirming that the cells of the glioma have a ΔΨm that is higher than a ΔΨm in non-cancerous glial cells when the level of TMRE fluorescence in the test sample is higher than the level of TMRE fluorescence in a control sample.

Similarly, cells of a glioma that have a ΔΨm that is higher than a ΔΨm in cells of a glioma that is not suppressed by the F₁Fₒ ATP synthase c-subunit leak channel opener may be identifiable by an assay comprising:
incubating a test sample comprising cells of the glioma (e.g. glioma to be treated) in the presence of TMRE, wherein the TMRE is provided at a concentration of 10 nM in the test sample, wherein the test sample is incubated at 37° C.;
comparing a level of TMRE fluorescence (using excitation and emission wavelengths of 550 nm and 575 nm, respectively) in the test sample with a level of TMRE fluorescence in a control sample comprising cells of a glioma that is not suppressed by the leak channel opener; and
confirming that the cells of the glioma have a ΔΨm that is higher than a ΔΨm in cells of a glioma that is not suppressed by the leak channel opener when the level of TMRE fluorescence in the test sample is higher than the level of TMRE fluorescence in a control sample.

The level of TMRE fluorescence may suitably correlate with the level of ΔΨm; for example a level of TMRE fluorescence that is 10% higher in the test sample compared to the control sample may indicate a level of ΔΨm that is 10% higher in the test sample compared to the control sample.

The test sample preferably comprises glioma cells isolated from the patient to be treated.

It will be appreciated that the assay methods do not necessarily require measurement of absolute levels (e.g. concentrations) of a ΔΨm, unless it is desired, because relative values may be sufficient for many applications of the invention. Accordingly, the ΔΨm can be the (absolute) total ΔΨm, or it can preferably be a “relative” ΔΨm, e.g., the difference between the ΔΨm detected in a test sample (e.g. cells of the glioma) and a control sample (e.g. non-cancerous glial cells). In some embodiments, the ΔΨm may be expressed by its level in a sample, or by the level of a reagent that detects ΔΨm. For example, where a fluorescent indicator of ΔΨm (such as TMRE) is employed, ΔΨm may be expressed as a level of fluorescent signal from the indicator.

Any sample (e.g. test sample of control sample) described herein that comprises a glioma or glioma cells is preferably an isolated sample, e.g. isolated from a patient.

The term “suppressing” a glioma may be used synonymously with the term “treating” a glioma herein. The term “suppress” or “suppressing” (e.g. “treat” or “treating”) as used herein encompasses prophylactic treatment (e.g. to prevent onset of glioma) as well as corrective treatment (treatment of a subject already suffering from glioma). Preferably “suppress” or “suppressing” (e.g. “treat” or “treating”) as used herein means corrective treatment. The term “suppress” or “suppressing” (e.g. “treat” or “treating”) encompasses treating both the glioma and a symptom thereof. In some embodiments “treat” or “treating” refers to a symptom of glioma.

Therefore, an F₁Fₒ ATP synthase c-subunit leak channel opener may be administered to a subject in a therapeutically effective amount or a prophylactically effective amount. A “therapeutically effective amount” is any amount of the leak channel opener (e.g. medicament), which when administered alone or in combination to a subject for treating a glioma (or a symptom thereof) is sufficient to effect such treatment of the disorder, or symptom thereof. A “prophylactically effective amount” is any amount of the leak channel opener (e.g. medicament) that, when administered alone or in combination to a subject inhibits or delays the onset or reoccurrence of glioma (or a symptom thereof). In some embodiments, the prophylactically effective amount prevents the onset or reoccurrence of glioma entirely. “Inhibiting” the onset means either lessening the likelihood of glioma onset (or symptom thereof) or preventing the onset entirely.

The terms “subject”, “individual” and “patient” are used interchangeably herein to refer to a mammalian subject, preferably a human.

As outlined above, a glioma that is the therapeutic target of the F₁Fₒ ATP synthase c-subunit leak channel opener of the invention is a glioma that can be suppressed by inducing H⁺ leakage through the mitochondrial F₁Fₒ ATP Synthase c-subunit leak channel. The glioma may be a subtype (e.g. highly proliferative subtype) of glioblastoma, however other stages/classifications of glioma are also intended to be embraced. Further information on glioma and exemplary stages thereof is provided below.

Gliomas are generally classified by the type of cell that characterises tumours of the glioma, by grade (e.g. severity), and by location in the body.

When characterised by cell type, where the glioma is characterised based on the specific type of cell with which cells of the glioma share histological features (but not necessarily from which they originate), examples of glioma include:
an ependymoma;
an astrocytoma (e.g. glioblastoma multiforme);
an oligodendroglioma;
a brainstem glioma;
and optic nerve glioma;
a mixed glioma (e.g. oligoastrocytoma).

When characterised by grade, where the glioma is categorised according a grade determined by pathologic evaluation of the tumor (i.e. neuropathological evaluation and diagnostics of brain tumor specimens performed according to “WHO Classification of Tumours of the Central Nervous System”), examples of glioma include:
WHO grade I glioma (e.g. biologically benign gliomas, comparatively low risk);
WHO grade II glioma (e.g. low-grade glioma, exhibit benign tendencies but can increase in grade over time);
WHO grade III glioma (e.g. associated with histologic evidence of malignancy, including nuclear atypia/anaplasia and increased mitotic activity; have anaplastic histology and infiltrative capacity);
WHO grade IV glioma (e.g. tumor cells are mitotically active, necrosis-prone, and generally associated with neovascularity and infiltration of surrounding tissue, a propensity for craniospinal dissemination, and a rapid postoperative progression and fatal outcomes); includes glioblastoma multiforme.

When characterised by location, where the glioma is classified according to location, examples of glioma include:
glioma of the supratentorial region, e.g. region of the brain located above the tentorium, in the cerebrum;
glioma of the infratentorial region, e.g. a region of the brain located below the tentorium, in the cerebellum;
pontine glioma, e.g. glioma is located in the pons of the brainstem.

In a preferable embodiment, the glioma is glioblastoma multiforme.

Advantageously, it has been demonstrated that a F₁Fₒ ATP synthase c-subunit leak channel opener of the invention finds utility in suppressing glioma that is refractory to the current standard of care, temozolomide (TMZ). Without wishing to be bound by theory, it is hypothesised that the efficacy of a leak channel opener as claimed in treating TMZ-resistant cells is due to the mitochondria of TMZ-resistant cells being particularly efficient, e.g. due to exposure to chronic sublethal hypoxia which might cause TMZ resistance.

In a preferable embodiment, the glioma is TMZ-resistant. For example, the patient may be TMZ refractory.

The inventors have demonstrated a correlation between the efficacy of the leak channel opener of the invention and the proliferative capacity of target glioma cells, which is not glioma-grade specific (e.g. extends to highly proliferative glioma of any grade). Thus, the glioma cells which are the target of the leak channel opener described herein define a subgroup of glioma the proliferation of which is fuelled by aberrantly efficient mitochondria. These subtypes of glioma are particularly suitable targets of the leak channel opener. Such subtypes may conveniently be characterised relative to other glioma subtypes which are comparatively less proliferative, and thus suppressed to a lesser extent (or not at all) by a leak channel opener of the invention.

Thus, the leak channel opener of the invention preferably targets a glioma that may be referred to as being a “highly proliferative glioma”. The term “highly proliferative glioma” may refer to a glioma that proliferates at a higher rate than a glioma that is not suppressed by a leak channel opener of the invention. Additionally or alternatively, the term “highly proliferative glioma” may refer to a glioma that proliferates at a higher rate than a non-cancerous glial cell.

The skilled person is aware of suitable techniques for detecting a rate of proliferation in a glioma. Typically, the cell mass of the glioma (tumor) may be measured at a first time point and a second (subsequent) time point. The increase in cell mass at said second time point compared to said first time point is indicative of the rate of proliferation. When the increase is 10% higher than a corresponding increase in a control that employs a glioma that is not suppressed by a leak channel opener of the invention (or that employs non-cancerous glial cells), the glioma (e.g. “highly proliferative glioma”) may be said to be 10% percent more proliferative than cells of a glioma that is not suppressed by the leak channel opener (or non-cancerous glial cells).

In one embodiment, cells of the glioma (e.g. “highly proliferative glioma”) are at least 10% percent more proliferative than cells of a glioma that is not suppressed by the leak channel opener. For example, cells of the glioma may be at least 15%, or at least 20% more proliferative than cells of a glioma that is not suppressed by the leak channel opener. For example, cells of the glioma may be at least 25%, 50%, 100%, 150%, 200%, 250%, 300%, 350% or 400% (preferably at least 300%) more proliferative than cells of a glioma that is not suppressed by the leak channel opener.

Throughout this specification, the term “cells of a glioma that is not suppressed by the leak channel opener” means that said cells demonstrate substantially no decrease in proliferation post-contact with the leak channel opener of the invention. The term “substantially” as used here preferably means there is no statistically significant decrease in proliferation. Said decrease (which is not substantial) may be a decrease of less than 5%, 2%, 1% or 0.5%. More preferably, the term “cells of a glioma that is not suppressed by the leak channel opener” refers to cells that do not demonstrate any decrease in proliferation at all (i.e. the decrease in the level is 0%).

In one embodiment, glioma suppression may be increased by at least 10%, 20%, 30%, 40%, 50%, 60% or 70% (preferably at least 20%) in the presence of a leak channel opener of the invention when compared to glioma suppression in the absence of the leak channel opener. In some embodiments, glioma suppression may be increased by at least 80%, 90% or 100% in the presence of a leak channel opener of the invention when compared to glioma suppression in the absence of the leak channel opener. In some embodiments, glioma suppression may be increased by at least 125%, 150% or 200% in the presence of a leak channel opener of the invention when compared to glioma suppression in the absence of the leak channel opener.

The term “in the absence of the leak channel opener” may refer to glioma suppression in the patient pre-administration of the leak channel opener. Additionally or alternatively, the term “in the absence of the leak channel opener” may refer to glioma suppression in a subject that has not been administered the leak channel opener.

The leak channel opener may preferably suppress glioma (e.g. suppress glioma proliferation) by at least 20% compared to glioma proliferation pre-administration of the leak channel opener. For example, the leak channel opener may suppress glioma proliferation by at least 30%, 40%, 50%, 60%, 70%, 80% or 90% (preferably at least 70%) compared to glioma proliferation pre-administration of the leak channel opener.

In a preferable embodiment, the leak channel opener decreases the mitochondrial transmembrane potential (ΔΨm) in cells of the glioma.

In one embodiment, ΔΨm may be decreased by at least 10%, 20%, 30%, 40%, 50%, 60% or 70% (preferably at least 20%) in the presence of a leak channel opener of the invention when compared to ΔΨm in the absence of the leak channel opener. In some embodiments, ΔΨm may be decreased by at least 80%, 90% or 100% in the presence of a leak channel opener of the invention when compared to ΔΨm in the absence of the leak channel opener. In some embodiments, ΔΨm may be decreased by at least 125%, 150% or 200% in the presence of a leak channel opener of the invention when compared to ΔΨm in the absence of the leak channel opener.

The term “in the absence of the leak channel opener” may refer to ΔΨm in the patient pre-administration of the leak channel opener. Additionally or alternatively, the term “in the absence of the leak channel opener” may refer to ΔΨm in subject that has not been administered the leak channel opener.

The patient may be a patient that has been identified for treatment with the leak channel opener of the invention by: detecting the presence of glioma suppression in an isolated glioma sample (e.g. biopsy) from the patient subsequent to contact with the leak channel opener, compared to a control glioma sample that has not been contacted with the leak channel opener.

In one embodiment, the patient may have been identified for treatment with the leak channel opener by: detecting a reduction in a level of glioma proliferation in an isolated glioma sample (e.g. biopsy) from the patient subsequent to contact with the leak channel opener, compared to a level of glioma proliferation in a control glioma sample that has not been contacted with the leak channel opener.

In one embodiment, the methods or uses of the invention may comprise a/the control step. With reference to any embodiment or aspect described herein that refers to a control sample, the control may be performed either within (i.e. constituting a step of) or external to (i.e. not constituting a step of) the methods of the invention.

Preferably, the control is obtained externally to the method of the invention and accessed during a comparison step of the invention.

The skilled person understands that the control sample is preferably derived from the same sample type as the sample type that is being tested, thus allowing for an appropriate comparison between the two (or more) samples. Thus, by way of example, if the sample is derived from a first location of the brain, the control sample is preferably also derived from said first location of the brain. If the sample is a biopsy, then the control sample will preferably also be a biopsy (e.g. suitably a biopsy of the same tumor, albeit not contacted with candidate drug in vitro).

A method of the invention (e.g. in the context of either a “F₁Fₒ ATP synthase c-subunit leak channel opener for use in a method of suppressing glioma” or a “method of suppressing a glioma in a patient”) may comprise (prior to administration of the leak channel opener) a step of identifying the patient as a patient suitable for treatment with the leak channel opener by: detecting the presence of glioma suppression in an isolated glioma sample (e.g. biopsy) from the patient subsequent to contact with the leak channel opener, compared to a control glioma sample that has not been contacted with the leak channel opener.

In one embodiment, a method of the invention (e.g. in the context of either a “F₁Fₒ ATP synthase c-subunit leak channel opener for use in a method of suppressing glioma” or a “method of suppressing a glioma in a patient”) may comprise (prior to administration of the leak channel opener) a step of identifying the patient as a patient suitable for treatment with the leak channel opener by: detecting a reduction in a level of glioma proliferation in an isolated glioma sample (e.g. biopsy) from the patient subsequent to contact with the leak channel opener, compared to a level of glioma proliferation in a control glioma sample that has not been contacted with the leak channel opener.

As outlined above, the leak channel opener of the invention induces H⁺ leakage through the mitochondrial F₁Fₒ ATP Synthase c-subunit leak channel. Said term “induces H⁺ leakage” may be used interchangeably with the term “promotes H⁺ leakage” herein.

In one embodiment, the leak channel opener induces proton leakage through the F₁Fₒ ATP Synthase c-subunit leak channel that is at least 5% greater than proton leakage in the absence of the leak channel opener. For example, the leak channel opener may induce proton leakage through the F₁Fₒ ATP Synthase c-subunit leak channel that is at least 10%, 15%, 25%, 35%, 45%, 55%, 65%, 75%, 85%, or 95% (preferably at least 45%) greater than proton leakage in the absence of the leak channel opener.

In other words, the proton leakage through the F₁Fₒ ATP Synthase c-subunit leak channel (in the glioma) may be at least 5% greater in the presence of a leak channel opener of the invention, when compared to proton leakage in the absence of the leak channel opener. For example, the proton leakage through the F₁Fₒ ATP Synthase c-subunit leak channel may be at least 10%, 15%, 25%, 35%, 45%, 55%, 65%, 75%, 85%, or 95% (preferably at least 45%) greater than proton leakage in the absence of the leak channel opener.

Proton leakage through the F₁Fₒ ATP Synthase c-subunit leak channel may be measured by an assay comprising:
a. admixing a submitochondrial vesicle (SMV) preparation with the F₁Fₒ ATP Synthase c-subunit leak channel opener and 9-Amino-6-chloro-2-methoxyacridine (ACMA), to provide an admixture comprising:
   i. leak channel opener;
   ii. ACMA; and
   iii. SMV preparation;
b. contacting the admixture with ATP;
c. measuring a level of fluorescence from ACMA (e.g. using excitation and emission wavelengths of 410 nm and 483 nm, respectively) (e.g. wherein the level of fluorescence correlates with the level of proton leakage);
d. comparing the level of fluorescence at step c) with a level of fluorescence in a control admixture lacking the leak channel opener; and
e. confirming that the leak channel opener induces proton leakage when the level of fluorescence is at least 5% greater compared to the level of fluorescence in the control admixture.

Proton leakage through the F₁Fₒ ATP Synthase c-subunit leak channel may be measured by an assay comprising:
a. admixing a submitochondrial vesicle (SMV) preparation with the F₁Fₒ ATP Synthase c-subunit leak channel opener and 9-Amino-6-chloro-2-methoxyacridine (ACMA), to provide an admixture comprising:
   i. leak channel opener;
   ii. ACMA; and
   iii. SMV preparation;
b. contacting the admixture with ATP, wherein the presence of ATP promotes translocation of protons across the SMV membrane and into the internal space of the SMV(s);
c. detecting H⁺ external to the SMV(s) by measuring a level of fluorescence from ACMA (e.g. using excitation and emission wavelengths of 410 nm and 483 nm, respectively) (e.g. wherein the level of fluorescence correlates with the level of proton leakage);
d. comparing the level of fluorescence measured at step c) with a level of fluorescence for a control admixture lacking the leak channel opener; and
e. confirming that the leak channel opener induces proton leakage when the level of fluorescence is at least 5% greater compared to the level of fluorescence for the control admixture.

More particularly, proton leakage through the F₁Fₒ ATP Synthase c-subunit leak channel may preferably be measured by an assay comprising:
a. admixing a submitochondrial vesicle (SMV) preparation with the leak channel opener and 9-Amino-6-chloro-2-methoxyacridine (ACMA), to provide an admixture comprising:
   i. 5-10 μM leak channel opener;
   ii. 2 μM ACMA; and
   iii. 5 μg SMV (measured by μg total polypeptide of the SMV preparation);
b. incubating the admixture for 20 minutes;
c. contacting the admixture with ATP, to provide the admixture with 1 mM ATP and a volume of 40 μl;
d. measuring a level of fluorescence (using excitation and emission wavelengths of 410 nm and 483 nm, respectively) from ACMA (e.g. wherein the level of fluorescence correlates with the level of proton leakage);
e. comparing the level of fluorescence at step d) with a level of fluorescence in a control admixture lacking the leak channel opener; and
f. confirming that the leak channel opener induces proton leakage when the level of fluorescence is at least 5% greater compared to the level of fluorescence in the control admixture.

More particularly, proton leakage through the F₁Fₒ ATP Synthase c-subunit leak channel may preferably be measured by an assay comprising:
a. admixing a submitochondrial vesicle (SMV) preparation with the leak channel opener and 9-Amino-6-chloro-2-methoxyacridine (ACMA), to provide an admixture comprising:
   i. 5-10 μM leak channel opener;
   ii. 2 μM ACMA; and
   iii. 5 μg SMV (measured by μg total polypeptide of the SMV preparation);
b. incubating the admixture for 20 minutes;
c. contacting the admixture with ATP, to provide the admixture with 1 mM ATP and a volume of 40 μl, wherein the presence of ATP promotes translocation of protons across the SMV membrane and into the internal space of the SMV(s);
d. detecting H⁺ external to the SMV(s) by measuring a level of fluorescence (using excitation and emission wavelengths of 410 nm and 483 nm, respectively) from ACMA (e.g. wherein the level of fluorescence correlates with the level of proton leakage);
e. comparing the level of fluorescence measured at step d) with a level of fluorescence for a control admixture lacking the leak channel opener; and
f. confirming that the leak channel opener induces proton leakage when the level of fluorescence is at least 5% greater compared to the level of fluorescence for the control admixture.

Preferably, an assay described herein comprises confirming that the leak channel opener induces proton leakage when the level of fluorescence is at least 15%, 25%, 35%, 45%, 55%, 65%, 75%, 85%, or 95% (preferably at least 75%) greater compared to the level of fluorescence in the control admixture.

A method of the invention (e.g. in the context of either a “F₁Fₒ ATP Synthase c-subunit leak channel opener for use in a method of suppressing glioma” or a “method of suppressing a glioma in a patient”) may comprise (prior to administration of the leak channel opener) a step of identifying the leak channel opener as being suitable for treating a glioma patient, by an assay that measures proton (H+) leakage through the mitochondrial F₁Fₒ ATP Synthase c-subunit leak channel, the assay preferably comprising:
a. providing an admixture comprising:
   i. the leak channel opener;
   ii. a submitochondrial vesicle (SMV) preparation; and
   iii. an H⁺ probe (preferably ACMA) (e.g. wherein the H⁺ probe remains outside of the SMV(s), or wherein the H⁺ probe only detects H⁺ that is outside of the SMV(s));
b. contacting the admixture with ATP;
c. measuring a level of H⁺ outside of the SMV(s) via the H⁺ probe;
d. comparing the level of H⁺ at step c) with a level of H⁺ in a control admixture lacking the leak channel opener; and
   i. identifying the leak channel opener as a candidate anti-glioma drug, that induces proton (H+) leakage through the mitochondrial F₁Fₒ ATP Synthase c-subunit leak channel, when the level of H⁺ is higher compared to the level of H⁺ in the control admixture; or
   ii. identifying that the leak channel opener is not a candidate anti-glioma drug, that induces proton (H+) leakage through the mitochondrial F₁Fₒ ATP Synthase c-subunit leak channel, when the level of H⁺ is the same or lower compared to the level of H⁺ in the control admixture.

A method of the invention (e.g. in the context of either a “F₁Fₒ ATP Synthase c-subunit leak channel opener for use in a method of suppressing glioma” or a “method of suppressing a glioma in a patient”) may comprise (prior to administration of the leak channel opener) a step of identifying the leak channel opener as being suitable for treating a glioma patient, by an assay that measures proton (H+) leakage through the mitochondrial F₁Fₒ ATP Synthase c-subunit leak channel, the assay preferably comprising:
a. providing an admixture comprising:
   i. the leak channel opener;
   ii. a submitochondrial vesicle (SMV) preparation; and
   iii. an H⁺ probe (preferably ACMA) (e.g. wherein the H⁺ probe remains outside of the SMV(s), or wherein the H⁺ probe only detects H⁺ that is outside of the SMV(s));
b. contacting the admixture with ATP, wherein the presence of ATP promotes translocation of protons across the SMV membrane and into the internal space of the SMV;
c. detecting H+ external to the SMV(s) with the H+ probe, and determining a value of H+ external to the SMV(s);
d. comparing the H⁺ level determined at step c) with that of a corresponding H⁺ level for a control admixture that lacks the candidate drug; and
   i. identifying the drug as a candidate anti-glioma drug that promotes H⁺ leakage, when the H⁺ level is higher compared to the H⁺ level for the control admixture; or
   ii. identifying that the drug is not a candidate anti-glioma drug that promotes H⁺ leakage, when the H⁺ level is the same or lower compared to the H⁺ level for the control admixture.

More particularly, a method of the invention may comprise (prior to administration of the leak channel opener) a step of identifying the leak channel opener as being suitable for treating a glioma patient, by an assay that measures proton (H+) leakage through the mitochondrial F₁Fₒ ATP Synthase c-subunit leak channel, the assay comprising:
a. admixing a submitochondrial vesicle (SMV) preparation with the leak channel opener and 9-Amino-6-chloro-2-methoxyacridine (ACMA), to provide an admixture comprising:
   i. leak channel opener (preferably 5-10 μM leak channel opener, more preferably about 10 μM leak channel opener);
   ii. ACMA (preferably 0.5-5 μM ACMA, more preferably about 2 μM ACMA); and
   iii. SMV (preferably 2-5 μg SMV, more preferably about 5 μg SMV);
b. incubating the admixture for at least 20 minutes (preferably for 20 minutes);
c. contacting the admixture with ATP, to provide the admixture with ATP (preferably 0.1-10 mM ATP, more preferably about 1 mM ATP), and preferably a volume of 30-50 μl (more preferably 40 μl);
d. measuring a level of fluorescence from ACMA (using excitation and emission wavelengths of 410 nm and 483 nm, respectively) (e.g. wherein the level of fluorescence correlates with the level of proton leakage);
e. comparing the level of fluorescence at step d) with a level of fluorescence in a control admixture lacking the leak channel opener; and
f. confirming that the leak channel opener induces proton leakage when the level of fluorescence is at least 5% greater compared to the level of fluorescence in the control admixture.

More particularly, a method of the invention may comprise (prior to administration of the leak channel opener) a step of identifying the leak channel opener as being suitable for treating a glioma patient, by an assay that measures proton (H+) leakage through the mitochondrial F₁Fₒ ATP Synthase c-subunit leak channel, the assay comprising:
a. admixing a submitochondrial vesicle (SMV) preparation with the leak channel opener and 9-Amino-6-chloro-2-methoxyacridine (ACMA), to provide an admixture comprising:
   i. leak channel opener (preferably 5-10 μM leak channel opener, more preferably about 10 μM leak channel opener);
   ii. ACMA (preferably 0.5-5 μM ACMA, more preferably about 2 μM ACMA); and
   iii. SMV (preferably 2-5 μg SMV, more preferably about 5 μg SMV);
b. incubating the admixture for at least 20 minutes (preferably for 20 minutes);
c. contacting the admixture with ATP, to provide the admixture with ATP (preferably 0.1-10 mM ATP, more preferably about 1 mM ATP), and preferably a volume of 30-50 μl (more preferably 40 μl), wherein the presence of ATP promotes translocation of protons across the SMV membrane and into the internal space of the SMV;
d. detecting H⁺ external to the SMV(s) by measuring a level of fluorescence from ACMA (using excitation and emission wavelengths of 410 nm and 483 nm, respectively) (e.g. wherein the level of fluorescence correlates with the level of proton leakage);
e. comparing the level of fluorescence measured at step d) with a level of fluorescence for a control admixture lacking the leak channel opener; and
f. confirming that the leak channel opener promotes proton leakage when the level of fluorescence is at least 5% greater compared to the level of fluorescence for the control admixture.

Preferably, an assay described herein comprises confirming that the leak channel opener induces proton leakage when the level of fluorescence is at least 15%, 25%, 35%, 45%, 55%, 65%, 75%, 85%, or 95% (preferably at least 75%) greater compared to the level of fluorescence in the control admixture.

Such step (that measures H+ leakage) may be performed prior to, e.g. in combination with, the above-mentioned step of identifying the patient as a patient suitable for treatment with the leak channel opener. For example, such “two-step” validation may be used to identity the most appropriate leak channel opener for any given patient in a personalised manner.

The “F₁Fₒ ATP Synthase c-subunit leak channel opener” of the invention (e.g. “medicament”) may be referred to as “a substance” or “composition”, and is a medicament, preferably a compound (e.g. a small molecule drug), although alternative drug types (e.g. antibodies) which target the mitochondrial F₁Fₒ ATP Synthase c-subunit leak channel (causing proton leakage) may also be embraced. The “F₁Fₒ ATP Synthase c-subunit leak channel opener” of the invention (e.g. “medicament”) is preferably a drug (e.g. small molecule drug). The terms “medicament” and drug may be used interchangeably herein.

Examples of a suitable F₁Fₒ ATP Synthase c-subunit leak channel opener of the invention include a compound selected from the group consisting of Donepezil (e.g. Donepezil-HCl), Salmeterol, Nitazoxanide, Efavirenz, Duloxetine (e.g. Duloxetine-HCl), Febuxostat, Colistin (e.g. Colistin Sulfate), Sulfadiazine, Clotrimazole, Dexchlorpheniramine (e.g. Dexchlorpheniramine Maleate), Hydroxyzine (e.g. Hydroxyzine Dihydrochloride), Procarbazine (e.g. Procarbazine-HCl), Mitoxantrone (e.g. Mitoxantrone-HCl), Amiodarone (e.g. Amiodarone-HCl), Dihydroergotamine (e.g. Dihydroergotamine Mesylate), Sertaconazole, Propranolol (e.g. Propranolol-HCl), Darifenacin (e.g. Darifenacin-HBr), Fluvoxamine (e.g. Fluvoxamine Maleate), Doxepin (e.g. Doxepin-HCl), Iloperidone, Telmisartan, Malathion, Acitretin, Tolterodine (e.g. Tolterodine Tartrate), Vinblastine (e.g. Vinblastine Sulfate), Dactinomycin (also known as Actinomycin D), Rifapentine, Irinotecan (e.g. Irinotecan-HCl), Gefitinib, Dasatinib, Amlodipine, Clomipramine (e.g. Clomipramine-HCl), Sunitinib (e.g. Sunitinib Malate), Loxapine (e.g. Loxapine Succinate), Perphenazine, Tamoxifen (e.g. Tamoxifen Citrate), Thioridazine (e.g. Thioridazine-HCl), and Cyproheptadine (e.g. Cyproheptadine-HCl Sesquihydrate); or a combination thereof.

For example, the F₁Fₒ ATP Synthase c-subunit leak channel opener may be a compound selected from the group consisting of Donepezil (e.g. Donepezil-HCl), Salmeterol, Colistin (e.g. Colistin Sulfate), Sulfadiazine, Dexchlorpheniramine (e.g. Dexchlorpheniramine Maleate), Hydroxyzine (e.g. Hydroxyzine Dihydrochloride), Sertaconazole, Iloperidone, Acitretin, Tolterodine (e.g. Tolterodine Tartrate), Rifapentine, Cyproheptadine (e.g. Cyproheptadine-HCL Sesquihydrate) or a combination thereof. The leak channel opener may be a compound selected from the group consisting of Colistin (e.g. Colistin Sulfate), Sulfadiazine, Dexchlorpheniramine (e.g. Dexchlorpheniramine Maleate), Hydroxyzine (e.g. Hydroxyzine Dihydrochloride), Sertaconazole, Iloperidone, Acitretin, Tolterodine (e.g. Tolterodine Tartrate), Rifapentine, Cyproheptadine (e.g. Cyproheptadine-HCL Sesquihydrate) or a combination thereof.

The F₁Fₒ ATP Synthase c-subunit leak channel opener may be a compound selected from the group consisting of Efavirenz, Dactinomycin (also known as Actinomycin D), Irinotecan (e.g. Irinotecan-HCl), Gefitinib, Clomipramine (e.g. Clomipramine-HCl), Sunitinib (e.g. Sunitinib Malate), Perphenazine, Tamoxifen (e.g. Tamoxifen Citrate), Thioridazine (e.g. Thioridazine-HCl); or a combination thereof.

In a preferable embodiment, the F₁Fₒ ATP Synthase c-subunit leak channel opener is a compound selected from the group consisting of Donepezil (e.g. Donepezil-HCl), Salmeterol, Nitazoxanide, Efavirenz, and Duloxetine (e.g. Duloxetine-HCl); or a combination thereof. The leak channel opener may be, for example, Donepezil (e.g. Donepezil-HCl), Salmeterol, or a combination thereof.

The invention embraces combination therapy, in which two or more F₁Fₒ ATP Synthase c-subunit leak channel openers (e.g. that target the mitochondrial F₁Fₒ ATP Synthase c-subunit leak channel as described herein) are administered. Said two or more leak channel openers may be administered simultaneously, separately or sequentially.

Additionally or alternatively, the F₁Fₒ ATP Synthase c-subunit leak channel of the invention may be administered in combination with TMZ (e.g. wherein the leak channel opener and TMZ are administered simultaneously, separately or sequentially).

The ability of candidate compounds to induce leakage may suitably be investigated via an assay that utilises a combination of submitochondrial vesicles and a proton probe, such as the fluorescent probe 9-Amino-6-chloro-2-methoxyacridine, ACMA.

A “submitochondrial vesicle” (SMV) is a vesicle formed from an isolated mitochondrial inner membrane (or fragment thereof) that comprises (e.g. is enriched in) F₁Fₒ ATP synthase protein complexes and preferably lacks mitochondrial outer membrane (where “lacks” preferably means “substantially lacks”). The term “submitochondrial vesicle” may be used synonymously with the term “F₁Fₒ ATP Synthase-enriched submitochondrial vesicle” or “F₁Fₒ ATP Synthase-comprising submitochondrial vesicle” (or in other words, “a submitochondrial vesicle that comprises F₁Fₒ ATP Synthase”).

Suitably, a “submitochondrial vesicle” may be referred to as a vesicle formed from an isolated inner membrane (or fragment thereof) of a mitochondria, wherein said inner membrane comprises an F₁Fₒ ATP synthase protein complex (preferably wherein said inner membrane is enriched in F₁Fₒ ATP synthase protein complex).

A “SMV preparation” refers to a preparation of SMVs isolated from a tissue sample. The quantity of SMV preparation used in a method of the invention is preferably defined by the total polypeptide content of the preparation. For example, where reference to 1 μg of SMV preparation is made, this preferably corresponds to 1 μg of polypeptide(s) measurable in the SMV preparation.

An SMV preparation is typically isolated from brain tissue to provide a SMV preparation, but may be isolated from alternative tissue such as liver tissue. SMVs are enriched in F₁Fₒ ATP synthase protein complexes (as the major proton pump), and the SMVs retain the structural and functional integrity of the F₁Fₒ ATP synthase, for example as shown in Alavian, K. N. et al (Nat Cell Biol 13, 1224-1233, doi:10.1038/ncb2330 (2011)) which is incorporated herein by reference. As such, SMVs provide a powerful system for investigating F₁Fₒ ATP synthase c-subunit leak channel activity. An SMV preparation is generally prepared from animal brain (e.g. rodent or swine brain), as described in Sacchetti, S. et al (J. Vis. Exp. (75), e4394, doi:10.3791/4394 (2013)) which is incorporated herein by reference; SMVs purified by this method are essentially free of contamination by other subcellular organelles as shown in FIG. 1 of said reference.

An SMV preparation may suitably be prepared by the following steps, and thus the SMV preparation may be defined as “an SMV preparation obtainable by a method comprising” the following steps:
1. mincing animal tissue (e.g. brain tissue) in 7 mL isolation buffer (comprising 250 mM sucrose, 20 mM HEPES pH 7.2, 1 mM EDTA, 0.5% BSA w/v) to provide minced tissue (mincing performed with a scissors);
2. homogenising the minced tissue to provide homogenised minced tissue, by subjecting the minced tissue to 100 strokes with a Teflon coated pestle, rotated at 500 rpm, using a beaker of ice-water to chill the homogenising vessel;
3. subjecting the homogenised minced tissue (0.5 g tissue per ml of isolation buffer) to centrifugation at 1,500× g, 4° C. for 10 mins, to provide a first supernatant (comprising cytosolic, ER, mitochondrial and synaptosomal fractions);
4. subjecting the first supernatant to centrifugation at 10,000× g, 4° C. for 15 mins, and resuspending the pellet (comprising mitochondrial and synaptosome fractions) in about 500 μL isolation buffer, to provide a cellular fraction of mitochondria and synaptosomes;
5. subjecting the cellular fraction of mitochondria and synaptosomes to pressurisation at 1,200 psi (˜83 bar) with N₂ gas, for 10 mins, followed by immediate release of pressure; wherein said pressurisation provides a post-pressurisation cellular fraction of mitochondria and synaptosomes;
6. placing the post-pressurisation cellular fraction of mitochondria and disrupted synaptosomes on a Ficoll gradient comprising a bottom layer of 10% Ficoll diluted in isolation buffer, and a top layer of 7.5% Ficoll diluted in isolation buffer, and performing centrifugation at 126,500× g, 4° C. for 20 mins to provide a pellet comprising purified mitochondria;
7. resuspending the pellet comprising purified mitochondria in isolation buffer, centrifuging at 16,000× g, 4° C. for 10 mins, and resuspending the pellet in isolation buffer to provide washed purified mitochondria;
8. admixing the washed purified mitochondria with an equal volume of 1% digitonin, on ice, for 15 mins, to provide an admixture comprising digitonin and washed purified mitochondria;
9. subjecting the admixture comprising digitonin and washed purified mitochondria to centrifugation at 16,000× g, 4° C. for 10 mins, resuspending the pellet in isolation buffer and admixing with 1/100 dilution of 20% Lubrol PX (C12E9) solution to provide an admixture comprising Lubrol PX, and incubating on ice for 15 mins;
10. layering the admixture comprising Lubrol PX onto isolation buffer and centrifuging at 182,000× g, 4° C. for 60 mins, and resuspending the pellet in isolation buffer to provide a sample comprising SMVs; and
11. subjecting the sample comprising SMVs to centrifugation at 16,000× g, 4° C. for 10 mins, and resuspending the pellet in isolation buffer to provide a SMV preparation.

An SMV preparation may be prepared either within (i.e. constituting a step of) or external to (i.e. not constituting a step of) the methods of the invention. Preferably, the SMV preparation is prepared externally to the methods of the invention and obtained during a step of contacting a reagent (e.g. drug, leak channel opener, ACMA etc) with the SMV preparation.

A “proton (H⁺ ) probe” as described herein means a reagent that can be used to detect and quantify a level of protons outside of an SMV(s), for example a proton level within a buffer in which the SMV(s) is suspended. Suitably, the H⁺ probe may remain outside of the SMV. Additionally or alternatively, the H⁺ probe may only detect H⁺ that is outside of the SMV. In other words, it may be said that the proton probe does not translocate across the SMV membrane, into the internal space of the SMV(s). In yet other words, it may be said that the proton probe remains excluded from the internal space of the SMV(s). A suitable H⁺ probe is ACMA.

ACMA is a fluorescent H⁺ probe, having excitation/emission maxima of ˜419/483 nm (respectively) that interacts with protons and can be used to monitor proton movement. ACMA is advantageously SMV-excluded (e.g. does not translocate across the membrane to the internal space of the SMV) and thus suited to probing H⁺ being pumped out of the SMVs. ACMA is fluorescent only in the presence of protons. Thus, a level of ACMA fluorescence correlates with the level of proton leakage (e.g. correlates with or is indicative of a level of proton leakage). While ACMA is a preferred H⁺ probe, the present disclosure embraces alternative H⁺ probes that may be employed to measure H⁺ in a method of the invention.

An exemplary schematic outlining the principles of a suitable assay is shown in FIG. 3. In an exemplary summary, an SMV preparation is incubated in buffer comprising the H⁺ probe (ACMA), in the presence of a leak channel opener/drug under investigation (or in the absence of the drug in control experiments). After the addition of ATP to the SMVs, ATPase activity results in decreased H⁺ concentration in the buffer (e.g. due to movement of protons into the SMVs in response to ATP hydrolysis). In the control experiments (where the c-subunit remains closed or substantially closed) this leads to a rapid and sustained drop in signal from the H⁺ probe (e.g. drop in the level of fluorescence from ACMA). However, where the leak channel is opened (e.g. due to the action of a candidate anti-glioma drug that induces channel opening), the drop in signal is shallower and transient as protons are pumped back outside of the SMVs via the c-subunit leak channel and thus detectable in the buffer again via the signal from the H⁺ probe. The inventors have demonstrated that such screen can be utilised to identify drugs that increase leak currents in the membrane by opening of the leak channel/s, reducing the ATP quenching effect (see Example 2).

In a further aspect, the invention provides a method for identifying a candidate anti-glioma drug that is a mitochondrial F₁Fₒ ATP Synthase c-subunit leak channel opener (e.g. that induces proton (H⁺) leakage through a mitochondrial F₁Fₒ ATP Synthase c-subunit leak channel), the method comprising:
a. providing an admixture comprising:
   i. a drug;
   ii. a submitochondrial vesicle (SMV) (e.g. SMV preparation); and
   iii. an H⁺ probe, wherein the H⁺ probe remains outside of the SMV(s), or wherein the H⁺ probe only detects H⁺ that is outside of the SMV(s);
b. contacting the admixture with ATP;
c. measuring a level of H⁺ outside of the SMV(s) via the H⁺ probe;
d. comparing the level of H⁺ at step c) with a level of H⁺ in a control admixture lacking the drug; and
   i. identifying the drug as a candidate anti-glioma drug that is a mitochondrial F₁Fₒ ATP Synthase c-subunit leak channel opener (e.g. that induces H⁺ leakage through the mitochondrial F₁Fₒ ATP Synthase c-subunit leak channel), when the level of H⁺ is higher compared to the level of H⁺ in the control admixture; or
   ii. identifying that the drug is not a candidate anti-glioma drug that is a mitochondrial F₁Fₒ ATP Synthase c-subunit leak channel opener (e.g. that induces H⁺ leakage through the mitochondrial F₁Fₒ ATP Synthase c-subunit leak channel), when the level of H⁺ is the same or lower compared to the level of H⁺ in the control admixture.

An aspect provides a method for identifying a candidate anti-glioma drug that promotes proton (H⁺) leakage through a mitochondrial F₁Fₒ ATP Synthase c-subunit leak channel, the method comprising:
a. providing an admixture comprising:
   i. a candidate drug;
   ii. a submitochondrial vesicle (SMV) preparation; and
   iii. an H⁺ probe;
b. contacting the admixture with adenosine triphosphate (ATP), wherein the presence of ATP promotes translocation of protons across the SMV membrane and into the internal space of the SMV;
c. detecting H⁺ external to the SMV(s) with the H⁺ probe, and determining a value of H⁺ external to the SMV(s);
d. comparing the H⁺ level determined at step c) with that of a corresponding H⁺ level for a control admixture that lacks the candidate drug; and
   i. identifying the drug as a candidate anti-glioma drug that promotes H⁺ leakage, when the H⁺ level is higher compared to the H⁺ level for the control admixture; or
   ii. identifying that the drug is not a candidate anti-glioma drug that promotes H⁺ leakage, when the H⁺ level is the same or lower compared to the H⁺ level for the control admixture.

The invention embraces corresponding use of an assay that measures proton (H+) leakage through the mitochondrial F₁Fₒ ATP Synthase c-subunit leak channel, for identifying a candidate anti-glioma drug that is a mitochondrial F₁Fₒ ATP Synthase c-subunit leak channel opener.

An aspect of the invention provides use of an assay that measures proton (H⁺) leakage through the mitochondrial F₁Fₒ ATP Synthase c-subunit leak channel, for identifying a candidate anti-glioma drug (e.g. that is a mitochondrial F₁Fₒ ATP Synthase c-subunit leak channel opener), the assay comprising:
a. providing an admixture comprising:
   i. a drug;
   ii. a submitochondrial vesicle (SMV) (e.g. SMV preparation); and
   iii. an H⁺ probe, wherein the H⁺ probe remains outside of the SMV(s), or wherein the H⁺ probe only detects H⁺ that is outside of the SMV(s);
b. contacting the admixture with ATP;
c. measuring a level of H⁺ outside of the SMV(s) via the H⁺ probe;
d. comparing the level of H⁺ at step c) with a level of H⁺ in a control admixture lacking the drug; and
   i. identifying the drug as a candidate anti-glioma drug that is a mitochondrial F₁Fₒ ATP Synthase c-subunit leak channel opener (e.g. that induces H⁺ leakage through the mitochondrial F₁Fₒ ATP Synthase c-subunit leak channel), when the level of H⁺ is higher compared to the level of H⁺ in the control admixture; or
   ii. identifying that the drug is not a candidate anti-glioma drug that is a mitochondrial F₁Fₒ ATP Synthase c-subunit leak channel opener (e.g. that induces H⁺ leakage through the mitochondrial F₁Fₒ ATP Synthase c-subunit leak channel), when the level of H⁺ is the same or lower compared to the level of H⁺ in the control admixture.

An aspect provides use of an assay that measures proton (H+) leakage through the mitochondrial F₁Fₒ ATP Synthase c-subunit leak channel, for identifying a candidate anti-glioma drug, the assay comprising:
a. providing an admixture comprising:
   i. a candidate drug;
   ii. a submitochondrial vesicle (SMV) preparation; and
   iii. an H⁺ probe;
b. contacting the admixture with ATP, wherein the presence of ATP promotes translocation of protons across the SMV membrane and into the internal space of the SMV;
c. detecting H⁺ external to the SMV(s) with the H⁺ probe, and determining a value of H⁺ external to the SMV(s);
d. comparing the H⁺ level determined at step c) with that of a corresponding H⁺ level for a control admixture that lacks the candidate drug; and
   i. identifying the drug as a candidate anti-glioma drug that promotes H⁺ leakage, when the H⁺ level is higher compared to the H⁺ level for the control admixture; or
   ii. identifying that the drug is not a candidate anti-glioma drug that promotes H⁺ leakage, when the H⁺ level is the same or lower compared to the H⁺ level for the control admixture.

For any method or use for identifying a candidate anti-glioma drug described herein, preferably steps a) to e) are repeated for at least one further candidate drug. Indeed, the invention can advantageously be employed as part of a drug screen, for identifying one or more candidate drugs from a pool of compounds such as a compound library.

For example, steps a) to e) may be repeated for at least 2, 4, 6, 8, 10, 12, 14, 16, 20, 22, 24, 26, 30, 32, 34, 36, 38, or 40 further candidate drugs. For example, steps a) to e) may be repeated for at least 10, 15, 20, 25, 30, 35, 40, 45, 50, 55, 60, 65, 70, 75, 80, 85, 90, 95 or 100 further candidate drugs. For example, steps a) to e) may be repeated for at least 50, 75, 100, 125, 150, 175, 200, 225, 250, 275, 300, 325, 350, 375, 400, 425, 450, 475 or 500 further candidate drugs.

The H⁺ probe of the invention is preferably a compound or molecule, such as ACMA.

The term “the H⁺ probe remains outside of the SMV” means that the H⁺ probe does not move into the inner space of the SMV (e.g. does not translocate across the SMV membrane), and thus only detects protons outside of the SMV. Thus, the H⁺ probe is preferably a probe that does not translocate across the SMV membrane (e.g. and thus only detects protons outside of the SMV). In other words, such H⁺ probe is SMV-excluded. As such, it is possible to focus proton detection in a buffer in which the SMVs are suspended and into which protons leak (via the c-subunit leak channel). By way of example (as described above), ACMA is SMV-excluded, and displays signal only in the presence of proton, thus allowing detection of the proton level external to the SMV. For example, ACMA fluorescence signal drops when protons are pumped into the SMV (because ACMA does not enter the SMV with the protons) and rises again when protons leak out of the SMV.

The term “external to the SMV(s)” may refer to a buffer (or composition) in which the SMV(s) is suspended. Thus, it may be said that the proton probe detects proton in the buffer (or composition) in which the SMV(s) is suspended.

It will be appreciated that the assay methods do not necessarily require measurement of absolute levels (e.g. concentrations) of a proton, unless it is desired, because relative values may be sufficient for many applications of the invention. Accordingly, the “level” or “concentration” can be the (absolute) total level/concentration of protons detected in a sample, or it can preferably be a “relative” level/concentration, e.g., the difference between the proton level detected in a test sample and e.g. a control sample. In some embodiments, the proton level may be expressed by its level in a sample, or by the level of a reagent (H⁺ probe) that detects protons. For example, where the H⁺ probe is a fluorescent marker, a proton level may be expressed as a level of fluorescent signal from the probe.

The H⁺ probe is preferably 9-Amino-6-chloro-2-methoxyacridine (ACMA).

The inventors have demonstrated a strong correlation between the ability of a drug to induce proton (H+) leakage and to suppress glioma proliferation in highly proliferative glioma populations (see Example 2 and FIG. 4). Thus, a drug demonstrated to induce H⁺ leakage through the mitochondrial F₁Fₒ ATP Synthase c-subunit leak channel may advantageously be identified as a “candidate anti-glioma drug”. Methods/uses of the invention may include one or more further validation step (e.g. step to confirm the candidate drug's ability to suppress a glioma).

A method or use of the invention may further comprise (e.g. subsequent to identifying the drug as a candidate anti-glioma drug) a step of identifying a candidate anti-glioma drug as suitable for use as an anti-glioma drug by:
a. contacting an isolated glioma sample with the candidate anti-glioma drug, and incubating the sample;
b. detecting the presence or absence of glioma suppression when compared to a control glioma sample incubated without the candidate drug; and
   i. identifying the candidate drug as suitable for use as an anti-glioma drug when suppression is detected; or
   ii. identifying the candidate drug as unsuitable for use as an anti-glioma drug when suppression is not detected.

The sample may be any sample that comprises glioma cells. For example, the sample may be a glioma cell line. The sample may be a patient sample, for example a biopsy sample comprising tissue extracted from a region (e.g. of the brain) where the glioma is present. Thus, the sample may be an isolated sample obtained from a patient.

Throughout this disclosure, e.g. in any aspect or embodiment that refers to glioma suppression, the presence of glioma suppression may be detected when suppression is increased, compared to the control glioma sample, preferably by at least 5%, 10%, 15%, 20%, 25%, 30%, 35%, 40%, 45%, 50%, 60%, 70%, 80%, 90%, 100%, 110%, 120%, 130%, 140% or 150%. Said increase in suppression is preferably statistically significant. In some embodiments, said increase may be identified by a fold change of the level of suppression. In one embodiment, an increase may be at least about 1.1-fold, 1.2-fold, 1.25-fold or 1.5-fold greater when compared to the control glioma sample.

In one embodiment, a method or use of the invention may further comprise (e.g. subsequent to identifying the drug as a candidate anti-glioma drug) a step of identifying a candidate anti-glioma drug as suitable for use as an anti-glioma drug by:
a. contacting an isolated glioma sample with the candidate anti-glioma drug, and incubating the sample;
b. detecting the presence or absence of a reduction in a level of glioma proliferation in the isolated glioma sample, when compared to a level of glioma proliferation in a control sample that has not been contacted with the candidate drug; and
   i. identifying the candidate drug as suitable for use as an anti-glioma drug when reduced proliferation is detected; or
   ii. identifying the candidate drug as unsuitable for use as an anti-glioma drug when reduced proliferation is not detected.

Throughout this disclosure, e.g. in any aspect or embodiment that refers to glioma proliferation, the term “reduced proliferation is not detected” means that substantially no reduction in proliferation is detected. The term “substantially” as used herein in the context of the term “reduced proliferation is not detected” preferably means there is no statistically significant reduction in proliferation. Said reduction (which is not substantial) may be a reduction of less than 5%, 2%, 1% or 0.5%, preferably less than 0.1%. More preferably, the term “reduced proliferation is not detected” as used herein means that the level of proliferation is not reduced at all (i.e. the reduction in the level of proliferation is 0%).

The skilled person is aware of suitable methodologies to measure proliferation of a glioma. For example, the size of a (isolated) glioma tumor may be measured, suitably wherein the difference between size at a first time point and size at a second (later) timepoint is indicative of a level of glioma proliferation. Additionally or alternatively, the number of cells in the isolated sample may be quantified, suitably wherein the difference between the number of cells at a first time point and the number of cells at a second (later) timepoint is indicative of a level of glioma proliferation.

A further aspect of the invention provides a screening method for identifying an anti-glioma drug, the method comprising:
a. obtaining a candidate drug that is a mitochondrial F₁Fₒ ATP Synthase c-subunit leak channel opener (e.g. that induces proton leakage through the mitochondrial F₁Fₒ ATP Synthase c-subunit leak channel); preferably wherein the candidate drug has been identified as a candidate anti-glioma drug that is a mitochondrial F₁Fₒ ATP Synthase c-subunit leak channel opener by a method described herein;
b. contacting an isolated glioma sample with the candidate drug, and incubating the sample;
c. detecting the presence or absence of glioma suppression when compared to a control glioma sample incubated without the candidate drug; and
   i. identifying the candidate drug as suitable for use as an anti-glioma drug when suppression is detected; or
   ii. identifying the candidate drug as unsuitable for use as an anti-glioma drug when suppression is not detected.

A further aspect of the invention provides a screening method for identifying an anti-glioma drug, the method comprising:
a. obtaining a candidate drug that is a mitochondrial F₁Fₒ ATP Synthase c-subunit leak channel opener (e.g. that induces proton leakage through the mitochondrial F₁Fₒ ATP Synthase c-subunit leak channel); preferably wherein the candidate drug has been identified as a candidate anti-glioma drug that induces H⁺ leakage through a mitochondrial F₁Fₒ ATP Synthase c-subunit leak channel by a method described herein;
b. contacting an isolated glioma sample with the candidate drug, and incubating the sample;
c. detecting the presence or absence of a reduction in a level of glioma proliferation in the isolated glioma sample, when compared to a level of glioma proliferation in a control sample that has not been contacted with the candidate drug; and
   i. identifying the candidate drug as suitable for use as an anti-glioma drug when reduced proliferation is detected; or
   ii. identifying the candidate drug as unsuitable for use as an anti-glioma drug when reduced proliferation is not detected.

The invention also finds utility in personalised medicine, allowing an optimal treatment to be identified for any given patient, for example by identifying a leak channel opener (whether comprising a single active ingredient of a combination of active ingredients) that optimally suppresses proliferation of an isolated glioma sample (e.g. biopsy) from the patient.

Advantageously, pre-screening for ‘responder patients’ (e.g. for responsiveness to treatment with a leak channel opener that targets the mitochondrial F₁Fₒ ATP Synthase c-subunit leak channel described herein) allows for a more effective method of treating a patient having a glioma. Other benefits associated with such methods are evident to the skilled person, for example treating only responsive patients allows for more cost-efficient and/or economical prescribing of medication. Alternatively or additionally, patient prognosis may be improved by way of early and/or effective treatment with a leak channel opener that is identified as being suitable for treating the glioma in said patient (e.g. by inducing proton leakage in glioma cells).

In one aspect, the invention provides a method (e.g. in vitro method) for identifying a glioma patient's suitability for treatment with a mitochondrial F₁Fₒ ATP Synthase c-subunit leak channel opener (e.g. a medicament that targets the mitochondrial F₁Fₒ ATP Synthase c-subunit leak channel), preferably wherein said medicament was identified by a method or use described herein for identifying a candidate anti-glioma drug that promotes proton (H⁺) leakage through a mitochondrial F₁Fₒ ATP Synthase c-subunit leak channel, the method comprising:
a. contacting an isolated glioma sample obtained from the patient with a mitochondrial F₁Fₒ ATP Synthase c-subunit leak channel opener (preferably wherein the leak channel opener has been identified as a candidate anti-glioma drug that is a mitochondrial F₁Fₒ ATP Synthase c-subunit leak channel opener by a method described herein), and incubating the sample;
b. detecting the presence or absence of glioma suppression when compared to a control glioma sample incubated without the leak channel opener; and
   i. identifying the patient as being suitable for treatment with the leak channel opener when suppression is detected; or
   ii. identifying the patient as being unsuitable for treatment with the leak channel opener when suppression is not detected.

An aspect provides a method (e.g. in vitro method) for identifying a glioma patient's suitability for treatment with a medicament comprising a mitochondrial F₁Fₒ ATP Synthase c-subunit leak channel opener (preferably wherein said medicament was identified by a method or use described herein for identifying a candidate anti-glioma drug that promotes proton (H⁺) leakage through a mitochondrial F₁Fₒ ATP Synthase c-subunit leak channel), the method comprising:
a. contacting an isolated glioma sample obtained from the patient with the medicament comprising F₁Fₒ ATP Synthase c-subunit leak channel opener, and incubating the sample;
b. detecting the presence or absence of glioma suppression when compared to a control glioma sample incubated in the absence of the medicament comprising leak channel opener; and
   i. identifying the patient as being suitable for treatment with the medicament comprising leak channel opener when suppression is detected; or
   ii. identifying the patient as being unsuitable for treatment with the medicament comprising leak channel opener when suppression is not detected.

A yet further aspect of the invention provides a method (e.g. in vitro method) for identifying the suitability of a mitochondrial F₁Fₒ ATP Synthase c-subunit leak channel opener (e.g. medicament that targets the mitochondrial F₁Fₒ ATP Synthase c-subunit leak channel) for treating a glioma patient, the method comprising:
a. contacting an isolated glioma sample with a mitochondrial F₁Fₒ ATP Synthase c-subunit leak channel opener (preferably wherein the leak channel opener has been identified as a candidate anti-glioma drug that is a mitochondrial F₁Fₒ ATP Synthase c-subunit leak channel opener by a method described herein), and incubating the sample;
b. detecting the presence or absence of glioma suppression when compared to a control glioma sample incubated without the leak channel opener; and
   i. identifying the leak channel opener as being suitable for treating glioma when suppression is detected; or
   ii. identifying the leak channel opener as being unsuitable for treating glioma when suppression is not detected.

An aspect provides a method (e.g. in vitro method) for identifying the suitability of medicament comprising a mitochondrial F₁Fₒ ATP Synthase c-subunit leak channel opener for treating a glioma patient (preferably wherein said medicament was identified by a method or use described herein for identifying a candidate anti-glioma drug that promotes proton (H⁺) leakage through a mitochondrial F₁Fₒ ATP Synthase c-subunit leak channel), the method comprising:
a. contacting an isolated glioma sample with the medicament comprising a mitochondrial F₁Fₒ ATP Synthase c-subunit leak channel opener, and incubating the sample;
b. detecting the presence or absence of glioma suppression when compared to a control glioma sample incubated without the medicament comprising leak channel opener; and
   i. identifying the leak channel opener as being suitable for treating glioma when suppression is detected; or
   ii. identifying the leak channel opener as being unsuitable for treating glioma when suppression is not detected.

Examples of suitable gliomas are described above. For example, the glioma may be GBM. As described above, the glioma may be TMZ resistant.

An “F₁Fₒ ATP Synthase c-subunit leak channel opener” (which may be referred to as a “medicament that targets the F₁Fₒ ATP Synthase c-subunit leak channel”) may advantageously have been confirmed to induce (aka promote) proton leakage through the F₁Fₒ ATP Synthase c-subunit leak channel, for example by an assay described herein.

In one embodiment, a F₁Fₒ ATP Synthase c-subunit leak channel opener (e.g. medicament that targets the F₁Fₒ ATP Synthase c-subunit leak channel) may have been confirmed to induce (aka promote) proton leakage through the F₁Fₒ ATP Synthase c-subunit leak channel by an assay comprising:
a. admixing a submitochondrial vesicle (SMV) (e.g. SMV preparation) with the leak channel opener and 9-Amino-6-chloro-2-methoxyacridine (ACMA), to provide an admixture comprising:
   i. leak channel opener;
   ii. ACMA; and
   iii. SMV (e.g. SMV preparation);
b. contacting the admixture with ATP;
c. measuring a level of fluorescence from ACMA (e.g. using excitation and emission wavelengths of 410 nm and 483 nm, respectively) (e.g. wherein the level of fluorescence correlates with the level of proton leakage);
d. comparing the level of fluorescence at step c) with a level of fluorescence in a control admixture lacking the leak channel opener; and
e. confirming that the leak channel opener induces proton leakage when the level of fluorescence is higher (for example, at least 5% higher) compared to the level of fluorescence in the control admixture.

In one embodiment, the leak channel opener or the candidate drug has been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
   i. leak channel opener or candidate drug;
   ii. ACMA; and
   iii. SMV preparation;
b. contacting the admixture with ATP, wherein the presence of ATP promotes translocation of protons across the SMV membrane and into the internal space of the SMV;
c. detecting H⁺ external to the SMV(s) by measuring a level of fluorescence (using excitation and emission wavelengths of 410 nm and 483 nm, respectively) from ACMA;
d. comparing the level of fluorescence measured at step c) with a level of fluorescence for a corresponding control admixture lacking the leak channel opener or candidate drug; and
e. confirming that the leak channel opener or candidate drug promotes proton leakage when the level of fluorescence is higher (for example, at least 5% higher) compared to the level of fluorescence for the control admixture.

More particularly (and preferably), a F₁Fₒ ATP Synthase c-subunit leak channel opener (e.g. medicament that targets the F₁Fₒ ATP Synthase c-subunit leak channel) may have been confirmed to induce proton leakage through the F₁Fₒ ATP Synthase c-subunit leak channel by an assay comprising:
a. admixing a submitochondrial vesicle (SMV) (e.g. SMV preparation) with the leak channel opener and 9-Amino-6-chloro-2-methoxyacridine (ACMA), to provide an admixture comprising:
   i. 5-10 μM leak channel opener;
   ii. 2 μM ACMA; and
   iii. 5 μg SMV (e.g. SMV preparation);
b. incubating the admixture for 20 minutes;
c. contacting the admixture with ATP, to provide the admixture with 1 mM ATP and a volume of 40 μl;
d. measuring a level of fluorescence from ACMA (using excitation and emission wavelengths of 410 nm and 483 nm, respectively) (e.g. wherein the level of fluorescence correlates with the level of proton leakage);
e. comparing the level of fluorescence at step d) with a level of fluorescence in a control admixture lacking the leak channel opener; and
f. confirming that the leak channel opener induces proton leakage when the level of fluorescence is higher (for example, at least 5% higher) compared to the level of fluorescence in the control admixture.

More particularly (and preferably), the leak channel opener or the candidate drug may have been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
   i. 5-10 μM leak channel opener or candidate drug;
   ii. 2 μM ACMA; and
   iii. 5 μg SMV;
b. incubating the admixture for 20 minutes;
c. contacting the admixture with ATP, to provide the admixture with 1 mM ATP and a volume of 40 μl, wherein the presence of ATP promotes translocation of protons across the SMV membrane and into the internal space of the SMV;
d. detecting H⁺ external to the SMV(s) by measuring a level of fluorescence (using excitation and emission wavelengths of 410 nm and 483 nm, respectively) from ACMA;
e. comparing the level of fluorescence at step d) with a level of fluorescence for a control admixture lacking the leak channel opener or candidate drug; and
f. confirming that the leak channel opener or candidate drug promotes proton leakage when the level of fluorescence is higher (for example, at least 5% higher) compared to the level of fluorescence for the control admixture.

Suitably, a method or use of the present invention may further comprise the step of recording on a suitable data carrier, the data obtained in said method or use.

Methods of the invention may further comprising administering the leak channel opener or the candidate drug to a glioma patient when:
a. the patient is identified as being suitable for treatment with the leak channel opener;
b. the leak channel opener is identified as being suitable for treating a glioma in a patient;
c. the candidate drug is identified as being suitable for use as an anti-glioma drug; or
d. the drug is identified as a candidate anti-glioma drug.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a submitochondrial vesicle” includes a plurality of such vesicles and reference to “the drug” includes reference to one or more drugs and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples, in which:

FIG. 1 shows the effect of hypoxia and mitochondrial metabolism on proliferation of primary glioma cells. A) Hypoxia decreases the rate of proliferation of primary cells from glioma with a high proliferation rate (grade IV (GBM) cells), while it has no effect on the proliferation of primary cells from glioma with a comparatively low proliferation rate (grade III cells). B) Inhibition of pyruvate entry into the mitochondria by UK5099 or inhibition of oxidative phosphorylation by rotenone or oligomycin decreases the proliferation of Grade IV (GBM) cells.

FIG. 2 shows correlation of mitochondrial membrane potential with proliferation of primary glioma cells. The mitochondrial membrane potential (measured by TMRE) is significantly higher in the primary cells from glioma with a high proliferation rate (Grade IV (GBM) cells, left), than that of the primary cells from glioma with a comparatively low proliferation rate (Grade III cells, right), demonstrating a correlation between the rate of proliferation and mitochondria activity. TMRE=tetramethylrhodamine, ethyl ester (a cell-permeant, cationic, red-orange fluorescent dye that is readily sequestered by active mitochondria).

FIG. 3 shows a schematic of the ACMA assay. The H+ fluorescent indicator 9-Amino-6-chloro-2-methoxyacridine (ACMA) and pig brain submitochondrial vesicles (SMVs) enriched in mitochondrial F₁Fₒ ATP synthase are used to measures the movement of protons into the SMVs in response to ATP hydrolysis. After the addition of ATP to the SMVs, ATPase activity results in decreased H+ concentration in the buffer solution surrounding the vesicles measured by a decrease in fluorescence intensity of the SMV-excluded H+ indicator, ACMA. The screen identified drugs that increase leak currents in the membrane and opening of the leak channel/s, reducing the ATP quenching effect.

FIG. 4 shows the effect of the ACMA screen-identified FDA approved drugs on the mitochondrial inner membrane leak currents and the proliferation of primary cells from glioma with a high proliferation rate (GBM cells). A) The positive and negative regulators of the mitochondrial leak channels identified using the FDA-approved drug library. Negative values represent reduction in ATP response as a result of drug treatment (@ 10 μM n=4 p<0.01). B) the effect of hits from the ACMA screen on proliferation of primary cells from glioma with a high proliferation rate (GBM cells) (treatment for 5 days @10 μM). C) Correlation of the effect of the FDA-approved drugs on leak channel opening and proliferation (Pearson correlation coefficient r=0.465). D, E) An example of the effect of the drugs on two primary cells lines from glioma with a high proliferation rate (GBM cell lines), one resistant to TMZ (D) and another that was responsive to TMZ (E).

### EXAMPLES

### Materials and Methods

### Protocol for Isolation of Mitochondria and Preparation of Submitochondrial Vesicles (SMVs) From Pig Brains

### Chemicals:

### Isolation Buffer (IB):

| | Final Concentration | | Volume in 500 mL | |
|---|---|---|---|---|
| 1M Sucrose | 250 | mM | 125 | mL |
| 1M HEPES (pH 7.2) | 20 | mM | 10 | mL |
| 0.1M EDTA | 1 | mM | 5 | mL |

| | | | |
|---|---|---|---|
| 5% BSA | 0.5% | 50 | mL |
| ddH₂O | — | 310 | mL |

500 mL bottle; filter and store at 4° C.

### Ficoll Centrifugation Buffer (FB):

| | Final Concentration | | Volume (for 2 tubes) | |
|---|---|---|---|---|
| 20% Ficoll-400 | 12.79% | | 22 | mL |
| 1M Sucrose | 349 | mM | 12 | mL |
| 1M Tris-HCl (pH 7.4) | 11 | mM | 375 | μL |
| 0.1M EDTA | 55 | μM | 18.75 | μL |

50 mL tube; Store at 4° C.

### Protocol:

1. Isolation Buffer (IB) and Ficoll Centrifugation Buffer (FB) were prepared from stock solutions and were stored at 4° C. or on ice on the day of use.
2. Whole pig brains were placed in chilled IB in a petri dish on ice to rinse off blood.
3. Brains were transferred to a second petri dish with 7 mL IB for mincing (with scissors) and then transferred to 10 mL homogenising vessel chilled on ice. Minced tissue was subjected to 100 strokes with a Teflon coated pestle, rotated at 500 rpm, using a beaker of ice-water to chill the homogenising vessel during this step.
4. SPIN 1 & 2: Homogenate (with 0.5 g tissue per ml of isolation buffer) was transferred to a 50 mL tube, making up to 25 mL with IB, and centrifugated at 1,500× g, 4° C. for 10 mins. The supernatant was recovered (“S1”; contains cytosolic, ER, mitochondrial and synaptosomal fractions) to a 50 ml tube and was kept on ice. The pellet was resuspended (undisrupted cells, nuclei and cell debris) with 5 mL IB for a second round of homogenisation (repeat steps 4-5).
5. SPIN 3: The S1 supernatants were pooled and split between two 50 mL tubes (made up to 50 mL with IB) and centrifugated at 10,000× g, 4° C. for 15 mins. The number of tubes was increased depending on the number of batches. The pellet(s) (“P2”; mitochondrial and synaptosome fractions) were resuspended in 250 μL IB (final volume should be ˜500 μL) and transferred to 1.5 ml tube(s).
6. The 1.5 mL tube(s) were placed upright and un-capped in the high-pressure vessel (max. of 6 tubes). The vessel was pressurised to 1,200 psi (˜83 bar) with N₂ gas. When charged, the vessel was sealed and incubated on ice for 15-20 mins.
7. During this step, the Ficoll gradient was prepared by diluting the FB with IB:

| | | |
|---|---|---|
| Ficoll % | 10% | 7.5% |

| | | | | |
|---|---|---|---|---|
| IB | 2.5 | mL | 6 | mL |
| FB | 15.5 | mL | 10 | mL |

in a pair of 13 mL ultracentrifuge tubes, 6 mL of the 10% solution was aliquoted before layering 5 mL of the 7.5% solution on top.
8. Quickly release the pressure in the vessel was quickly released; the 1.5 ml tube(s) were retrieved and the samples were pooled and mixed and placed onto the Ficoll gradient.
9. SPIN 4 (ULTRACENTRIFUGATION): The balanced 12 mL tubes were placed in the sample buckets, sealed and placed into the TH-641 rotor and centrifugated at 126,500× g, 4° C. for 20 mins.
10. Following the spin, the 7.5% Ficoll layer (including the top layer), the synaptosome layer at the 7.5%/10% gradient boundary and the 10% Ficoll layer were removed.
11. SPIN 5 (WASH): The pellets of purified mitochondria were resuspended in 100 μL IB, using a chilled 3 mL glass pestle. The suspension was triturated, transferred to a 1.5 mL tube and centrifugated at 16,000× g, 4° C. for 10 mins.
12. The supernatant was discarded, and the pellet(s) were re-suspended in IB at 5× the biomass then incubated with an equal volume of 1% digitonin, on ice, for 15 mins. The samples were mixed at 5 min intervals.
13. SPIN 6 & 7 (WASH): The solution(s) were made up to 1 mL with IB and centrifugated at 16,000× g, 4° C. for 10 mins. The supernatant was discarded, and the pellets were resuspended in 1 mL IB and centrifugated at 16,000× g, 4° C. for 10 mins.
14. The supernatant was discarded, and the pellets were resuspended in IB at 5× the biomass then incubated with a 1/100 dilution of 20% Lubrol PX (C12E9) solution, on ice for 15 mins. The samples were mixed at 5 min intervals.
15. SPIN 8 (ULTRACENTRIFUGATION): The sample(s) were layered onto the IB and centrifugated at 182,000× g, 4° C. for 60 mins.
16. SPIN 9 (WASH): The supernatant was discarded, and the pellet of submitochondrial vesicles (SMVs) were resuspended in 100 μL IB, using the chilled 3 mL glass pestle to break up the pellet before triturating the suspension. The samples were centrifugated at 16,000× g, 4° C. for 10 mins.
17. The supernatant was discarded, and the pellet(s) were resuspended in IB at 5× the biomass. The samples were aliquoted in 50-100 μL volumes, snap frozen in liquid N₂ and stored at −80° C.

### Cells

The cell lines employed were low-passage lines, derived from tumour tissue. The cells were obtained from tumour tissue of a highly proliferative glioma (which was a grade IV/glioblastoma multiforme glioma). For comparison, cells were also obtained from a tumour tissue of a comparatively slow-growing glioma (which was a grade III glioma).

### ACMA (9-amino-6-chloro-2-methoxy acridine) Assay Protocol

### Equipment and Materials:

ACMA assay buffer (concentrations used throughout the assay):

| | | |
|---|---|---|
| HEPES•KOH | 10 | mM |
| MgCl₂ | 5 | mM |
| KCl | 100 | mM |

| | |
|---|---|
| pH (HCl) | 7.5 |

ACMA solution:
Stock solution of 1 mg/ml (3.87 mM) ACMA in 100% ethanol
Diluted to working solution (3 μM) in assay buffer
Aliquots of 10 μL to be added to wells

### Protocol:

1. Sub-mitochondrial vesicles (SMVs) isolated from pig brains were thawed and prepared to 0.17 μg/μL in ACMA assay buffer.
2. Drug solutions were added to 386-well plates at a final concentration of 10 μM.
3. Sub-mitochondrial vesicles (SMVs), isolated from pig brains, were thawed and prepared to 0.17 μg/μL in ACMA assay buffer. 30 μL of this solution (containing 5 μg of polypeptide, as measured via the QuickStart Bradford Protein Assay (Bio-Rad, UK)) was added to each well.
4. Stock solution of 1 mg/ml (3.87 mM) ACMA (9-amino-6-chloro-2-methoxyacridine) were diluted to working solution 3 μM in assay buffer. Aliquots of 10 μL were added to each well (final concentration of 0.75 μM).
5. The samples were mixed and incubated at room temperature for 20 mins.
6. The ACMA fluorescence was measured at 1 min intervals using a Tecan Infinite M200 plate reader—excitation 412 nm, emission 480 nm.
7. Working solution (40 mM) of ATP was prepared in assay buffer just prior to use and added at a final concentration of 1 mM to the wells, following the first 2 baseline recordings.
8. Fluorescence was measured quickly after addition of ATP for another 7 mins (1 min intervals).

### Protocol for Sulforhodamine B (SRB) Colorimetric Assay: Cell Density Measurement, Based on the Quantity of Cellular Protein Content

### Materials:

50% (wt/vol) trichloroacetic acid (TCA)

1% (vol/vol) acetic acid

0.4% SRB in 1% (vol/vol) acetic acid

10 mM Tris base solution (pH 10.5) (121.14 g/mol; 1.21 g in 1 litre)

### Assay:

1. Without removing the cell culture medium, gently add 50% cold TCA to each well (the final concentration of TCA should be 10%), and incubate the plates at 4° C. for 1 h.
   For 150 μL medium volume in each well, add 30 μL of 50% TCA.
2. Wash the plates 4 times with slow-running tap water. Then allow the plates to air-dry at RT.
   After fixing and drying, the plates can be stored indefinitely at RT.
3. Add 0.4% SRB solution to each well (e.g. 80 μL/well). Leave at room temperature for 1 h (no shaking) and then quickly rinse the plates 4 times with 1% acetic acid to remove unbound dye. Then allow the plates to dry at RT.
   Stained and dried plates can be stored indefinitely at RT.
4. Add 10 mM Tris base solution to each well (e.g. 80 μL/well) and shake the plate for 5 min to solubilize the protein-bound dye. Alternatively, if a shaker is not available, SRB can be solubilized after 30 min in 10 mM Tris base solution.
5. Measure the OD at 490 nm in a microplate reader. The suboptimal wavelengths should be 490-530 nm.

### Tetramethylrhodamine, Ethyl Ester, Perchlorate (TMRE) Assay

TMRE (Thermo Fisher Scientific, UK) was added directly to the GBM and astrocytoma cultures at a concentration of 10 nM. The cultures were then incubated for 20 min. at 37° C. and imaged, using a confocal microscope (Ex/Em=550/575 nm). The intensity of signal was quantified using ImageJ.

### Glioma Suppression Assay

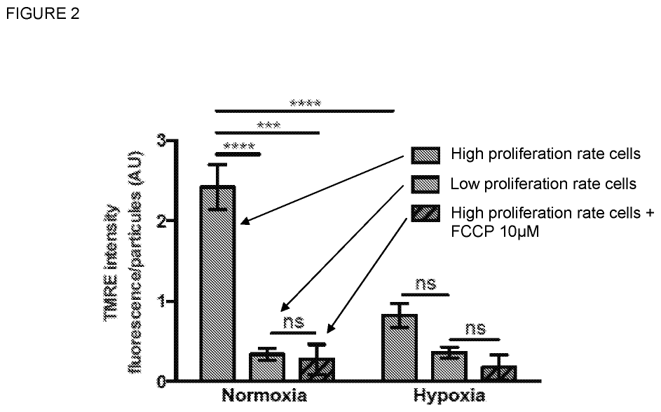

1000 Grade IV cells (cell line 1 in FIG. 2) were plated in cell culture medium (10% FBS in DMEM) per each well in 96-well plates. On day 1, the cells were treated with 10 μM of the indicated drugs and incubated at 37° C. for 5 days. SRB assay was performed prior to addition of the drugs and after 5 days. The values were normalized against those of the control (untreated cultures).

### EXAMPLE 1

### Demonstrating a Subgroup of Glioma Having Enhanced Mitochondrial Coupling and Efficiency in ATP Synthesis Through the Closure of the Inner Membrane Leak Channels

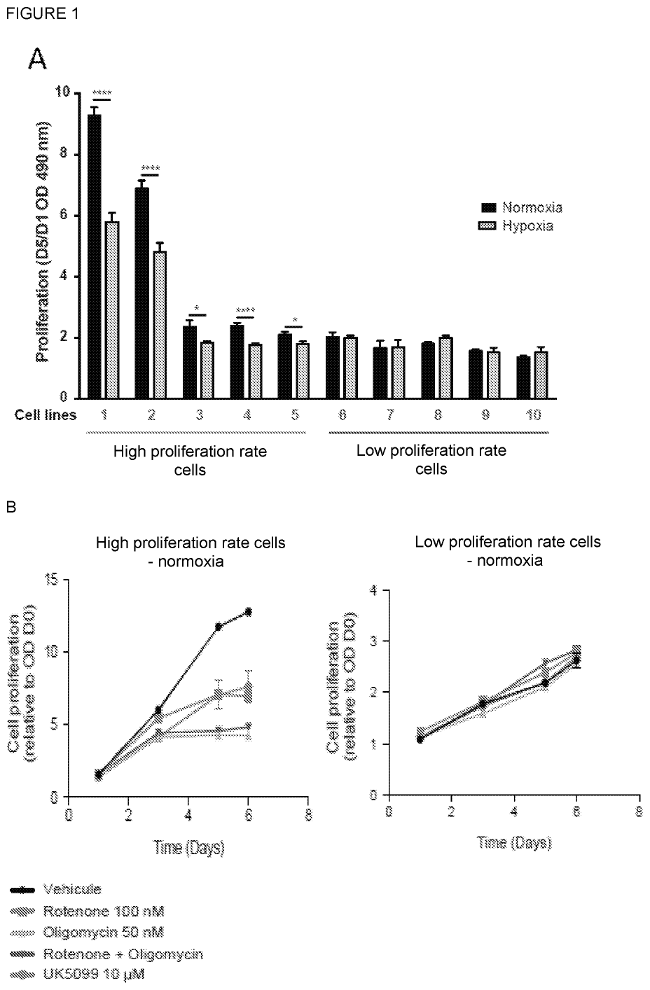

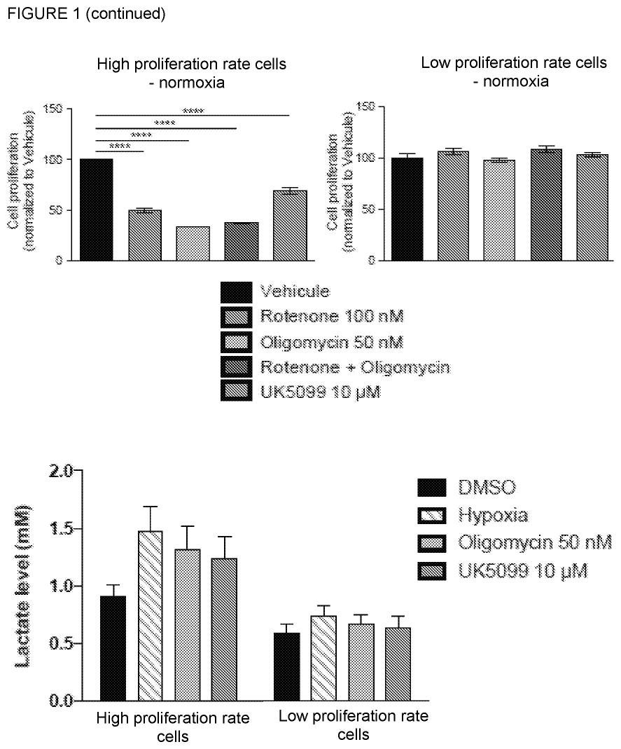

The inventors compared the metabolic and mitochondrial energetic profile of slow-growing glioma/astrocytoma cells (chosen in particular due to their relatively slow growth rate) to that in cells of a highly proliferative glioma primary tumour cells by performing ‘hypoxia’ experiments with primary tumour cells, in which primary tumour cells were deprived of adequate oxygen supply and compared with control cells not so deprived (‘normoxia’). The relatively lower rate of proliferation-cells were chosen as controls to demonstrate that higher efficacy of the drugs is seem in the more proliferative cells. The slow growing cells were selected from a grade III glioma, and the highly proliferative cells were chosen from a grade IV glioma i.e. glioblastoma multiforme cells. Interestingly, it was found that the rate of proliferation in glioblastoma multiforme (GBM, grade IV astrocytoma) primary tumour cells was significantly reduced under hypoxia, suggesting that they rely more on their mitochondria than the less proliferative glioma cells (FIG. 1A).

To further investigate this, primary tumour cells were incubated (both under hypoxia and normoxia) with the toxins rotenone (100 nM), oligomycin (50 nM), a combination of rotenone and oligomycin (100 nM and 50 nM, respectively) or UK5099 (10 μM). These toxins, which act by inhibiting mitochondrial respiratory complexes and oxidative phosphorylation, impeded the proliferation of GBM cells, without a significant effect on the grade III astrocytoma cells (FIG. 1B). Furthermore, a direct correlation was observed between mitochondrial membrane potential (measured by TMRE) and the rate of cellular proliferation, demonstrating that highly proliferative glioma cells employ efficient mitochondrial metabolism to achieve their higher proliferation rates (FIG. 2). These results demonstrate that enhanced mitochondrial coupling and efficiency in ATP synthesis through closure of the inner membrane leak channels contribute to the proliferation of high energy demand glioma cells.

### EXAMPLE 2

### Screening for Drugs That Induce Proton Leakage Through the F₁Fₒ ATP Synthase c-Subunit, and Thus Suppress Highly Proliferative Glioma Subgroups

The results of Example 1 suggest modulation of the mitochondrial leak channels (to induce proton leakage) as a therapeutic approach for treatment of highly proliferative glioma tumours. More particularly, it was postulated that inducing proton (H⁺) leakage through F₁Fₒ ATP synthase c-subunit may perturb the surprisingly high level of mitochondrial efficiency in this highly proliferative glioma subgroup. The inventors thus investigated whether modulation of this leak channel (inducing leakage) causes glioma suppression.

ACMA assay—Identifying Candidate Drugs

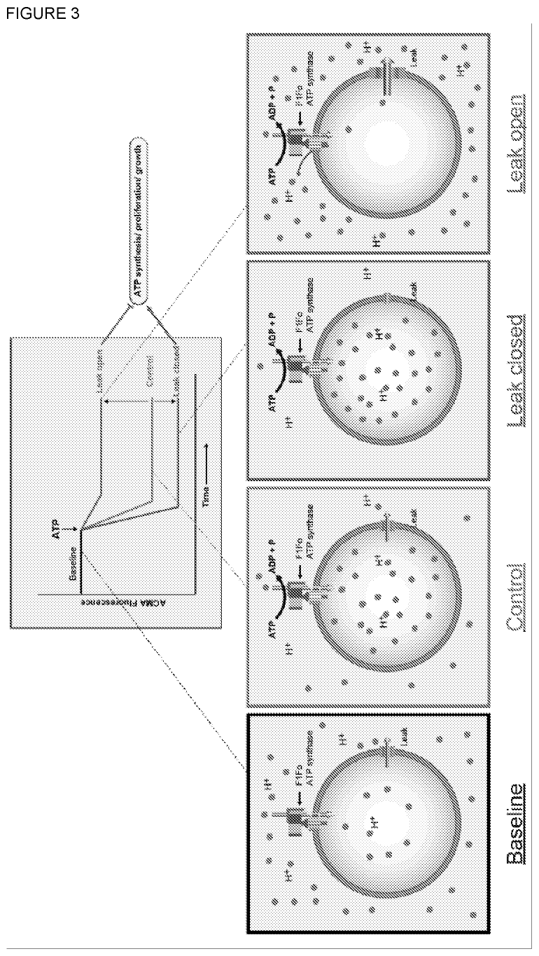

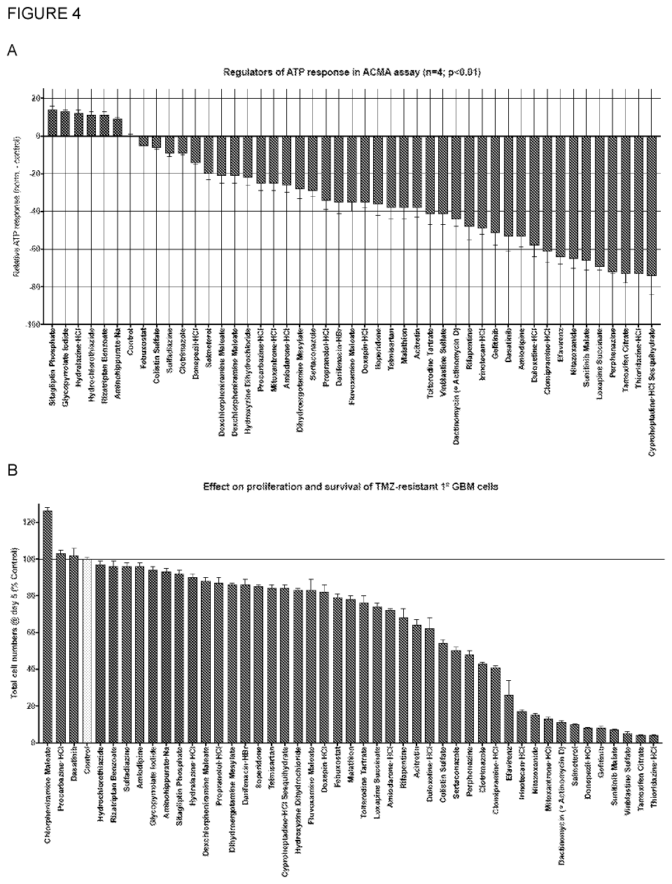

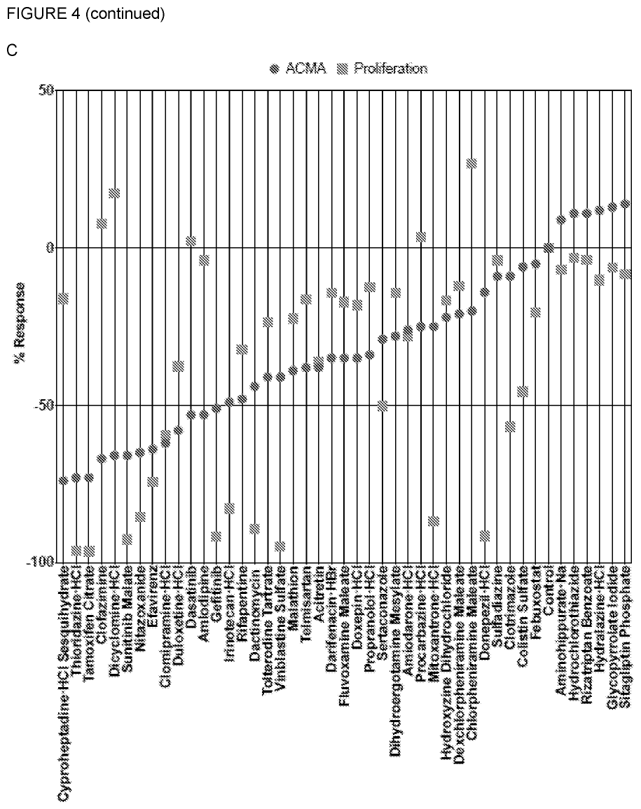

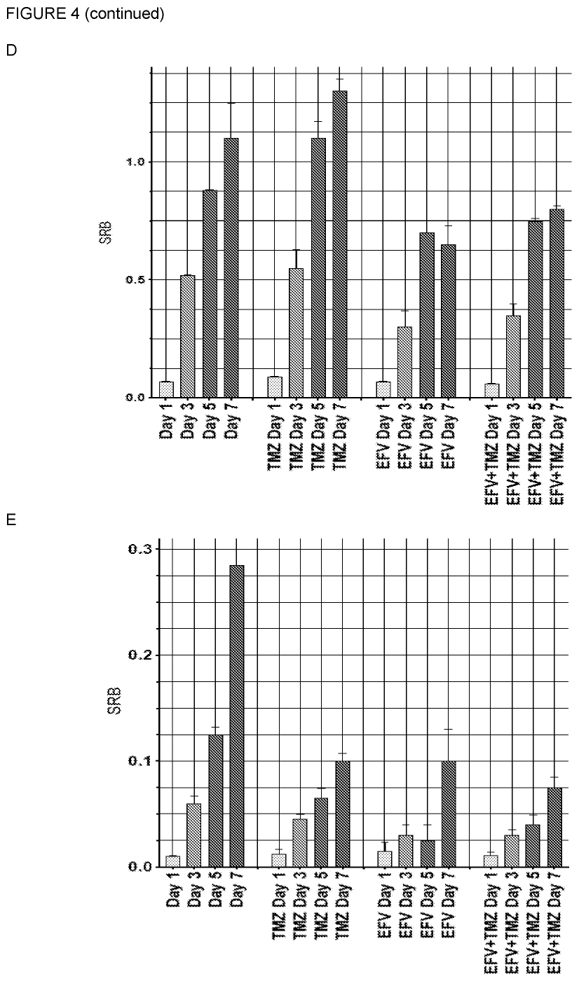

To investigate this, the inventors first conducted a screen to identify candidate anti-glioma drugs. In more detail, to identify the pharmacological agents that may reduce the efficiency of mitochondrial metabolic processes through increasing the ion leak currents associated with the F₁Fₒ ATP synthase (e.g. within the c-subunit), the inventors developed a high throughput fluorometric assay, using the H⁺ fluorescent indicator 9-Amino-6-chloro-2-methoxyacridine (ACMA) and isolated swine brain submitochondrial vesicles (SMVs), which are enriched for the F₁Fₒ ATP synthase. This assay measures the movement of H⁺ ions into the SMVs in response to ATP hydrolysis. After the addition of ATP to the SMVs, ATPase activity results in a decrease in the H⁺ concentration in the bath surrounding the vesicles which is measured by a decrease in fluorescence intensity of the SMV-excluded H⁺ indicator, ACMA (FIG. 3) (Alavian, K. N. et al .; Nat Cell Biol 13, 1224-1233, doi:10.1038/ncb2330 (2011). As negative control the ATP response was attenuated by either inhibition of H⁺ ion import through the Fₒ pump (by addition of oligomycin) or by leakage of H⁺ out of the SMVs (by carbonylcyanide p-trifluoromethoxyphenylhydrazone, FCCP). Using this assay, the effect of 786 drugs (approved by the FDA for clinical use) were tested, as well as a library of 12,000 new small molecules on the inner membrane leak current activity. The screen identified 40 FDA-approved drugs as regulators of proton leak conductance (n=4; p<0.01) (FIG. 4A).

### Glioma Suppression Assay

Having identified candidate anti-glioma drugs via the above-mentioned screen, the inventors proceeded to validate the suppressive effect of these drugs on isolated, patient-obtained glioma biopsies.

In more detail, the inventors examined the effect of the positive hits, as well as TMZ (the standard chemotherapeutic agent for treatment of adult and paediatric gliomas), on proliferation of GBM cells in vitro. In this hit confirmation assay, 35 of the 40 drugs reduced the proliferation of “GBM1” cells by 12-96% (n=4, p<0.01) (FIG. 4B). These results provided validation of the assay, as the vast majority of the drugs that attenuated the effect of ATP in the ACMA screen reduced the proliferation of GBM cells (Pearson correlation coefficient r=0.465) (FIG. 4C). Interestingly, TMZ was effective in slowing down proliferation of a subset of GBM cell lines and had no effect on the cell line (GBM1) with the highest rate of proliferation, while selected candidate drugs significantly reduced the rate of proliferation in this specific cell line (FIG. 4D,E). These results demonstrate that metabolic reprogramming through modulation of mitochondrial ion leak currents (as a standalone therapy and/or as part of a combination therapy) to be an effective method for treatment of malignant glioma. Advantageously, this two-step method (ACMA plus glioma suppression assays) allows identification of the most effective pharmacological agents and establishes a pathway for optimal treatment of GBM in future clinical trials.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for identifying a candidate anti-glioma drug that promotes proton (H⁺) leakage through a mitochondrial F₁Fₒ ATP Synthase c-subunit leak channel, the method comprising:
a. providing an admixture comprising:
i. a candidate drug;
ii. a submitochondrial vesicle (SMV) preparation; and
iii. an H⁺ probe;
b. contacting the admixture with adenosine triphosphate (ATP), wherein the presence of ATP promotes translocation of protons across the SMV membrane and into the internal space of the SMV(s);
c. detecting H⁺ external to the SMV(s) with the H⁺ probe, and determining a value of H⁺ external to the SMV(s);
d. comparing the H⁺ level determined at step c) with that of a corresponding reference H⁺ level for a control admixture that lacks the candidate drug; and
i. identifying the drug as a candidate anti-glioma drug that promotes H⁺ leakage, when the H⁺ level is higher compared to the reference H⁺ level for the control admixture; or
ii. identifying that the drug is not a candidate anti-glioma drug that promotes H⁺ leakage, when the H⁺ level is the same or lower compared to the reference H⁺ level for the control admixture.

2. Use of an assay that measures proton (H+) leakage through the mitochondrial F₁Fₒ ATP Synthase c-subunit leak channel, for identifying a candidate anti-glioma drug, the assay comprising:
a. providing an admixture comprising:
i. a candidate drug;
ii. a submitochondrial vesicle (SMV) preparation; and
iii. an H⁺ probe;
b. contacting the admixture with ATP, wherein the presence of ATP promotes translocation of protons across the SMV membrane and into the internal space of the SMV(s);
c. detecting H⁺ external to the SMV(s) with the H⁺ probe, and determining a value of H⁺ external to the SMV(s);
d. comparing the H⁺ level determined at step c) with that of a corresponding reference H⁺ level for a control admixture that lacks the candidate drug; and
i. identifying the drug as a candidate anti-glioma drug that promotes H⁺ leakage, when the H⁺ level is higher compared to the reference H⁺ level for the control admixture; or
ii. identifying that the drug is not a candidate anti-glioma drug that promotes H⁺ leakage, when the H⁺ level is the same or lower compared to the reference H⁺ level for the control admixture.

3. A method for identifying a glioma patient's suitability for treatment with a medicament comprising a mitochondrial F₁Fₒ ATP Synthase c-subunit leak channel opener (preferably wherein said medicament was identified by a method or use according to claims 1-2), the method comprising:
a. contacting an isolated glioma sample obtained from the patient with the medicament comprising F₁Fₒ ATP Synthase c-subunit leak channel opener, and incubating the sample;
b. detecting the presence or absence of glioma suppression when compared to a control glioma sample incubated in the absence of the medicament comprising leak channel opener; and
i. identifying the patient as being suitable for treatment with the medicament comprising leak channel opener when suppression is detected; or
ii. identifying the patient as being unsuitable for treatment with the medicament comprising leak channel opener when suppression is not detected.

4. A method for identifying the suitability of medicament comprising a mitochondrial F₁Fₒ ATP Synthase c-subunit leak channel opener for treating a glioma patient (preferably wherein said medicament was identified by a method or use according to claims 1-2), the method comprising:
a. contacting an isolated glioma sample with the medicament comprising a mitochondrial F₁Fₒ ATP Synthase c-subunit leak channel opener, and incubating the sample;
b. detecting the presence or absence of glioma suppression when compared to a control glioma sample incubated in the absence of the medicament comprising leak channel opener; and
i. identifying the leak channel opener as being suitable for treating glioma when suppression is detected; or
ii. identifying the leak channel opener as being unsuitable for treating glioma when suppression is not detected.

5. A screening method for identifying an anti-glioma drug, the method comprising:
a. obtaining a candidate drug that has been confirmed to be a mitochondrial F₁Fₒ ATP Synthase c-subunit leak channel opener (preferably by a method or use according to any of claims 1-2);
b. contacting an isolated glioma sample with the candidate drug, and incubating the sample;
c. detecting the presence or absence of glioma suppression when compared to a control glioma sample incubated in the absence of the candidate drug; and
i. identifying the candidate drug as suitable for use as an anti-glioma drug when suppression is detected; or
ii. identifying the candidate drug as unsuitable for use as an anti-glioma drug when suppression is not detected.

6. The method or use according to claim 1 or 2, wherein the H⁺ probe is 9-Amino-6-chloro-2-methoxyacridine (ACMA).

7. The method or use according to any one of claims 1-6, wherein the glioma is temozolomide (TMZ) resistant.

8. The method or use according to any one of claims 1-7, wherein the glioma is glioblastoma multiforme (GBM).

9. The method according to any one of claims 3-8, wherein the leak channel opener or the candidate drug has been confirmed to be a mitochondrial F₁Fₒ ATP Synthase c-subunit leak channel opener (e.g. has been confirmed to induce proton leakage through the F₁Fₒ ATP Synthase c-subunit leak channel) by an assay.

10. The method according to claim 9, wherein the leak channel opener or the candidate drug has been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
i. leak channel opener or candidate drug;
ii. ACMA; and
iii. SMV preparation;
b. contacting the admixture with ATP, wherein the presence of ATP promotes translocation of protons across the SMV membrane and into the internal space of the SMV;
c. detecting H⁺ external to the SMV(s) by measuring a level of fluorescence (using excitation and emission wavelengths of 410 nm and 483 nm, respectively) from ACMA;
d. comparing the level of fluorescence measured at step c) with a reference level of fluorescence for a corresponding control admixture lacking the leak channel opener or candidate drug; and
e. confirming that the leak channel opener or candidate drug promotes proton leakage when the level of fluorescence is higher (for example, at least 5% higher) compared to the reference level of fluorescence for the control admixture.

11. The method according to claim 9 or claim 10, wherein the leak channel opener or the candidate drug has been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
i. 5-10 μM leak channel opener or candidate drug;
ii. 2 μM ACMA; and
iii. 5 μg SMV;
b. incubating the admixture for 20 minutes;
c. contacting the admixture with ATP, to provide the admixture with 1 mM ATP and a volume of 40 μl, wherein the presence of ATP promotes translocation of protons across the SMV membrane and into the internal space of the SMV(s);
d. detecting H⁺ external to the SMV(s) by measuring a level of fluorescence (using excitation and emission wavelengths of 410 nm and 483 nm, respectively) from ACMA;
e. comparing the level of fluorescence measured at step d) with a reference level of fluorescence for a control admixture lacking the leak channel opener or candidate drug; and
f. confirming that the leak channel opener or candidate drug promotes proton leakage when the level of fluorescence is higher (for example, at least 5% higher) compared to the reference level of fluorescence for the control admixture.

12. The method or use according to any one of the preceding claims, further comprising administering the leak channel opener or the candidate drug to a glioma patient when:
a. the patient is identified as being suitable for treatment with the medicament comprising leak channel opener;
b. the leak channel opener is identified as being suitable for treating a glioma in a patient;
c. the candidate drug is identified as being suitable for use as an anti-glioma drug; or
d. the drug is identified as a candidate anti-glioma drug.

13. A medicament comprising a mitochondrial F₁Fₒ ATP Synthase c-subunit leak channel opener for use in a method of suppressing a glioma in a patient, by promoting:
proton (H⁺) leakage through the mitochondrial F₁Fₒ ATP Synthase c-subunit leak channel.

14. A method of suppressing a glioma in a patient by promoting proton (H+) leakage through the mitochondrial F₁Fₒ ATP Synthase c-subunit leak channel, the method comprising administering a medicament comprising a mitochondrial F₁Fₒ ATP Synthase c-subunit leak channel opener to the patient.

15. The medicament for use according to claim 13 or the method according to claim 14, wherein the glioma is temozolomide (TMZ) resistant.

16. The medicament for use or the method according to any one of claims 13-15, wherein the patient has been identified for treatment with the medicament comprising leak channel opener by:
detecting the presence of glioma suppression in an isolated sample from the patient subsequent to contact with the medicament comprising leak channel opener, compared to a corresponding control sample that has not been contacted with the leak channel opener.

17. The medicament for use or the method according to any one of claims 13-16, wherein the glioma is glioblastoma multiforme (GBM).

18. The medicament for use or the method according to any one of claims 13-17,
wherein cells of the glioma have a mitochondrial transmembrane potential (ΔΨm) that is higher than a mitochondrial transmembrane potential in cells of a glioma that is not suppressed by the medicament comprising leak channel opener, and
wherein the leak channel opener decreases the mitochondrial transmembrane potential in said cells of the glioma (e.g. by promoting H⁺ leakage through the mitochondrial F₁Fₒ ATP Synthase c-subunit leak channel).

19. The medicament for use or the method according to any one of claims 13-18, wherein the leak channel opener suppresses glioma growth by at least 30% compared to glioma growth pre-administration of the leak channel opener.

20. The medicament for use or the method according to any one of claims 13-19, wherein the leak channel opener promotes proton leakage through the F₁Fₒ ATP Synthase c-subunit leak channel that is at least 5% greater than proton leakage in the absence of the leak channel opener.

21. The medicament for use or method according to claim 20, wherein proton leakage through the F₁Fₒ ATP Synthase c-subunit leak channel is measurable (e.g. measured) by an assay comprising:
a. admixing an isolated submitochondrial vesicle (SMV) preparation with the leak channel opener and 9-Amino-6-chloro-2-methoxyacridine (ACMA), to provide an admixture comprising:
i. leak channel opener;
ii. ACMA; and
iii. SMV preparation;
b. contacting the admixture with ATP, wherein the presence of ATP promotes translocation of protons across the SMV membrane and into the internal space of the SMV(s);
c. detecting H⁺ external to the SMV(s) by measuring a level of fluorescence (e.g. using excitation and emission wavelengths of 410 nm and 483 nm, respectively) from ACMA in the admixture;
d. comparing the level of fluorescence measured at step c) with a reference level of fluorescence for a corresponding control admixture lacking the leak channel opener; and
e. confirming that the leak channel opener promotes proton leakage when the level of fluorescence is at least 5% greater compared to the reference level of fluorescence for the control admixture.

22. The medicament for use or method according to claim 20 or claim 21, wherein proton leakage through the F₁Fₒ ATP Synthase c-subunit leak channel is measurable (e.g. measured) by an assay comprising:
a. admixing an isolated submitochondrial vesicle (SMV) preparation with the leak channel opener and 9-Amino-6-chloro-2-methoxyacridine (ACMA), to provide an admixture comprising:
i. 5-10 μM leak channel opener;
ii. 2 μM ACMA; and
iii. 5 μg of a SMV preparation;
b. incubating the admixture for 20 minutes;
c. contacting the admixture with ATP, to provide the admixture with 1 mM ATP and a volume of 40 μl, wherein the presence of ATP promotes translocation of protons across the SMV membrane and into the internal space of the SMV(s);
d. detecting H⁺ external to the SMV(s) by measuring a level of fluorescence (using excitation and emission wavelengths of 410 nm and 483 nm, respectively) from ACMA;
e. comparing the level of fluorescence measured at step d) with a reference level of fluorescence from ACMA for a corresponding control admixture lacking the leak channel opener; and
f. confirming that the leak channel opener promotes proton leakage when the level of fluorescence is at least 5% greater compared to the reference level of fluorescence for the control admixture.

23. The medicament for use or the method according to any one of claims 13-22, wherein the leak channel opener is a compound selected from the group consisting of:
Donepezil-HCl, Salmeterol, Nitazoxanide, Efavirenz, Duloxetine-HCl, Febuxostat, Colistin Sulfate, Sulfadiazine, Clotrimazole, Dexchlorpheniramine Maleate, Hydroxyzine Dihydrochloride, Procarbazine-HCl, Mitoxantrone-HCl, Amiodarone-HCl, Dihydroergotamine Mesylate, Sertaconazole, Propranolol-HCl, Darifenacin-HBr, Fluvoxamine Maleate, Doxepin-HCl, Iloperidone, Telmisartan, Malathion, Acitretin, Tolterodine Tartrate, Vinblastine Sulfate, Dactinomycin (=Actinomycin D), Rifapentine, Irinotecan-HCl, Gefitinib, Dasatinib, Amlodipine, Clomipramine-HCl, Sunitinib Malate, Loxapine Succinate, Perphenazine, Tamoxifen Citrate, Thioridazine-HCl, and Cyproheptadine-HCl Sesquihydrate.

24. The medicament for use or the method according to any one of claims 13-23, wherein the leak channel opener is selected from the group consisting of:
Donepezil-HCl, Salmeterol, Nitazoxanide, Efavirenz, and Duloxetine-HCl.

25. The medicament for use or the method according to any one of the claims 13-24, wherein the method comprises administering at least two such leak channel opener(s) in combination.

26. The medicament for use or the method according to any one of claims 13-25, further comprising administering temozolomide.

27. The medicament for use, or the method according to any one of claims 13-26, wherein the leak channel opener or the candidate drug has been confirmed to be a mitochondrial F₁Fₒ ATP Synthase c-subunit leak channel opener (e.g. has been confirmed to induce proton leakage through the F₁Fₒ ATP Synthase c-subunit leak channel) by an assay.

28. The medicament for use, or the method according to claim 27, wherein the leak channel opener or the candidate drug has been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
i. leak channel opener or candidate drug;
ii. ACMA; and
iii. SMV preparation;
b. contacting the admixture with ATP, wherein the presence of ATP promotes translocation of protons across the SMV membrane and into the internal space of the SMV(s);
c. detecting H⁺ external to the SMV(s) by measuring a level of fluorescence (using excitation and emission wavelengths of 410 nm and 483 nm, respectively) from ACMA;
d. comparing the level of fluorescence measured at step c) with a reference level of fluorescence for a corresponding control admixture lacking the leak channel opener or candidate drug; and
e. confirming that the leak channel opener or candidate drug promotes proton leakage when the level of fluorescence is higher (for example, at least 5% higher) compared to the reference level of fluorescence for the control admixture.

29. The medicament for use, or the method according to claim 27 or claim 28, wherein the leak channel opener or the candidate drug has been confirmed to be a mitochondrial F₁Fₒ ATP Synthase c-subunit leak channel opener (e.g. has been confirmed to promote proton leakage through the F₁Fₒ ATP Synthase c-subunit leak channel) by an assay comprising:
a. admixing an isolated submitochondrial vesicle (SMV) preparation with the leak channel opener or candidate drug and 9-Amino-6-chloro-2-methoxyacridine (ACMA), to provide an admixture comprising:
i. 5-10 μM leak channel opener or candidate drug;
ii. 2 μM ACMA; and
iii. 5 μg SMV;
b. incubating the admixture for 20 minutes;
c. contacting the admixture with ATP, to provide the admixture with 1 mM ATP and a volume of 40 μl, wherein the presence of ATP promotes translocation of protons across the SMV membrane and into the internal space of the SMV(s);
d. detecting H⁺ external to the SMV(s) by measuring a level of fluorescence (using excitation and emission wavelengths of 410 nm and 483 nm, respectively) from ACMA;
e. comparing the level of fluorescence at step d) with a reference level of fluorescence for a control admixture lacking the leak channel opener or candidate drug; and
f. confirming that the leak channel opener or candidate drug promotes proton leakage when the level of fluorescence is higher (for example, at least 5% higher) compared to the reference level of fluorescence for the control admixture.
